SEQUENCE LISTING (15 sequences)

SEQ ID NO: 1 - DNA, 53 bp, Artificial Sequence
  Description of the artificial sequence Primer
gcgcatatgttaaaaaaagtttattattttttaatttttttatttattgttgc

SEQ ID NO: 2 - DNA, 54 bp, Artificial Sequence
  Description of the artificial sequence Primer
ggcctcgagttaattttccatttgcaaaacaaagttatcataagataccttgtc

SEQ ID NO: 3 - DNA, 20 bp, Artificial Sequence
  Description of the artificial sequence Primer
acccggaataagcagtcaag

SEQ ID NO: 4 - DNA, 20 bp, Artificial Sequence
  Description of the artificial sequence Primer
gctgccttacaagcctctac

SEQ ID NO: 5 - DNA, 20 bp, Artificial Sequence
  Description of the artificial sequence Primer
cagggctttctggtataggg

SEQ ID NO: 6 - DNA, 20 bp, Artificial Sequence
  Description of the artificial sequence Primer
ggatactttgcgtcgaaggc

SEQ ID NO: 7 - DNA, 22 bp, Artificial Sequence
  Description of the artificial sequence Primer
tgcatacttagcaccagcttca

SEQ ID NO: 8 - DNA, 42 bp, Artificial Sequence
  Description of the artificial sequence Primer
gatcgtcgacaagcaaggaggatatttttatgttaaaaaaag

SEQ ID NO: 9 - DNA, 52 bp, Artificial Sequence
  Description of the artificial sequence Primer
gagacctgcaggttaattttccatttgcaaaacaaagttatcataagatacc

SEQ ID NO: 10 - DNA, 20 bp, Artificial Sequence
  Description of the artificial sequence Primer
atgccatagcatttttatcc

SEQ ID NO: 11 - DNA, 20 bp, Artificial Sequence
  Description of the artificial sequence Primer
agtttatggcgggcgtcctg

SEQ ID NO: 12 - protein, 341 aa, Borreliella burgdorferi
MLKKVYYFLIFLFIVACSSSDDGKSEAKTVSLIVDGAFDDKGFNESSSKAIRKLKADLNINIIEKASTGNSYLGDIANLEDGNSNLIWGIGFRLSDILFQRASENVSVNYAIIEGVYDEIQIPKNLLNISFRSEEVAFLAGYFASKASKTGKIGFVGGVRGKVLESFMYGYEAGAKYANSNIKVVSQYVGTFGDFGLGRSTASNMYRDGVDIIFAAAGLSGIGVIEAAKELGPDHYIIGVDQDQSYLAPNNVIVSAVKKVDSLMYSLTKKYLETGVLDGGKTMFLGLKEDGLGLVLNENLKSNYSEIYNKSLKIGQSIMNGIIKVPYDKVSYDNFVLQMEN

SEQ ID NO: 13 - protein, 383 aa, Borreliella burgdorferi
MTISKNVFSKFILKFLNSSAFVSVFALFVGFLIVGLVVMGLGHSPFRMYFIILEIIFSSPKHLGYVLSYSAPLIFTGLSIGISLKAGLFNIGVEGQFILGSIVALIASVLLDLPPILHVITIFIITFLASGSLGILIGYLKAKFNISEVISGIMFNWILFHLNNIILDFSFIKRDNSDFSKPIKESAYIDFLASWKLSPEGLAYRSSHPFVNELLKAPLHFGIILGIIFAILIWFLLNKTIIGFKINATGSNIEASRCMGINVKAVLIFSMFLSAAVAGLAGAIQLMGVNKAIFKLSYMQGIGFNGIAASLMGNNSPIGIIFSSILFSILLYGSSRVQSLMGLPSSIVSLMMGIIVLVISASYFLNKIVLKGVKRVKYNNILD

SEQ ID NO: 14 - protein, 308 aa, Borreliella burgdorferi
MSNIIIFLISETLINSQTLILAGLGGLISEKSGIINIGLEGIMTIGAFSGATVAYFTNDPLFSIFAGGLAGLVLAILHAVFTIFLKSDQIITGMALNFLGPAIAVFISTLIFSSISTPPIEIKLPILFDGILNKTSFIFQIFGKRYSVYIAILSVVLFHIVFKYTKIGLRINASGENPEVLESVGVSVNKIRFFCVLLSGFLAGVSGAVLTTVVASSYVQGVTGGQGFIAIVMLIFGKWTPLGVLIGSFLFSFVKTLAIVLAQLPFFSLIMPPKMLVITPYLIIILSLIFFSKKNYAPKFLGITYKKH

SEQ ID NO: 15 - protein, 536 aa, Borreliella burgdorferi
MKEDVLVLENITKKYGDFVANDNVSIKFKAGEVHAILGENGAGKTTLMKTIYGIHQVNSGRIILKGQEINFKDSSEAIRNGIGMVFQHFMLIPQFTAVQNIILGYENSKFGFLDYKQARKKISSLSEKYGLKIDLEKRVEDLSVGMEQKIEILKVLYRNADIIIFDEPTAVLAPSEVDDFINILKVLAQEGHTVILITHKIKEIRSIAKKCTIMRLGKVVKTVNIADIDDKDLTKLMIGKEVALRSSKIKFENHFNILEIKNLSVKDERGVLKVKDVNLDLRNGEILGISGIEGSGQEDLVDAILGLKSIFKGDILKKNSSGNLESLKGLTIKQRIDKKIGNIPSDRQKHGLILEFNVMQNIGLKSFDNPDYLRLKTIHLKSNFDLKFNFFNFIKRQFNKFKKQFVGFDLNILRKLSNQLVSHFDIRPRDILSKVKHLSGGNQQKVIVAREISLEPDILLAIQPTRGLDVGAVENIYKRIIEQRDAGRSVLLVSLELDELVNVCDRIAVMHGGRIVGILEDNFDIDVIGKMMIGLS

(19) United States
(12) Patent Application Publication
(10) Pub. No.: US 2024/0082282 A1
(43) Pub. Date: Mar. 14, 2024

(54) Hygromycin A Compositions and Methods of Use

(71) Applicant: NORTHEASTERN UNIVERSITY, Boston, MA (US)

(72) Inventors: Anthony D'ONOFRIO, Northborough, MA (US); Kim LEWIS, Newton, MA (US)

(21) Appl. No.: 18/042,741

(22) PCT Filed: Sep. 1, 2021
(86) PCT No.: PCT/US21/48581

§ 371 Date: Feb. 23, 2023

Related U.S. Application Data
(60) Provisional application No. 63/226,357, filed on Jul. 28, 2021.
(60) Provisional application No. 63/073,073, filed on Sep. 1, 2020.

(51) Int. Cl. A61K 31/7048; A61P 31/04

(52) U.S. Cl. CPC A61K 31/7048; A61P 31/04

(57) ABSTRACT

This disclosure provides compounds and compositions useful for treating spirochete diseases such as Lyme disease as well as baits containing the compounds.

### CROSS REFERENCE TO RELATED APPLICATIONS

This application is a U.S. National Phase Application, filed under 35 U.S.C. § 371, of International Application No. PCT/US2021/048581, filed Sep. 1, 2021, which claims priority to, and the benefit of, U.S. Provisional Application No. 63/073,073, filed Sep. 1, 2020, and U.S. Provisional Application No. 63/226,357, filed Jul. 28, 2021. Each of the foregoing applications is incorporated herein by reference in its entirety.

### GOVERNMENT LICENSE RIGHTS

This invention was made with government support under grants RO1A1152210 and RO1A1122286. awarded by the National Institutes of Health. The government has certain rights in the invention.

### SEQUENCE LISTING

The present application is being filed along with a Sequence Listing in electronic format. The Sequence Listing is provided as a file entitled 21591011PCT.txt, created on Aug. 16, 2021, which is 16,354 bytes in size. The information in the electronic format of the sequence listing is incorporated herein by reference in its entirety.

### FIELD OF THE DISCLOSURE

The present disclosure relates to hygromycin A compositions and methods of using the compositions. In particular, methods and compositions related to the use of Hygromycin A for targeting Borreliella and Borreliella associated diseases are provided.

### BACKGROUND

Lyme disease is caused by a spirochete Borreliella burgdorferi (also referred to herein as Borrelia burgdorferi and/or B. burgdorferi). Although early antibiotic treatment is effective for most patients, between 10-20% of patients continue to have symptoms such as fatigue, muscle pain, and cognitive impairment long after therapy.

The incidence and geographic range of Lyme disease caused by B. burgdorferi have been increasing due to a variety of factors, including expansion of the habitat range of the tick vector, increased intersection of human domiciles and animal hosts of ticks, and longer seasonal activity due to climate change. Current estimates from the Centers of Disease Control suggest that there are almost half a million cases of Lyme disease in the U.S. per year (Schwartz, Emerg Infect Dis, 2021; herein incorporated by reference in its entirety). Acute infection is notable for a characteristic rash called erythema migrans, which starts at the site of inoculation by the tick. From there, the bacteria disseminate quickly to other skin sites, the heart, and the peripheral and central nervous system causing carditis, radiculitis/nerve palsies and meningitis. If not treated during the early phase of infection, late symptoms of infection with B. burgdorferi include arthritis and neurological issues.

The acute disease is treated with broad-spectrum antibiotics such as doxycycline, amoxicillin and ceftriaxone. Treatment with broad-spectrum compounds comes at a considerable cost, disrupting the gut microbiome and selecting for resistance in off-target bacteria. The microbiome shapes the immune system during development and contributes to maintaining a healthy GI tract and preventing cardiovascular, mental health and autoimmune diseases. For example, standard treatment for Lyme disease currently involves amoxicillin or ceftriaxone. These two antibiotics are known to significantly disrupt the gut microbiome of the individual being treated. Therapies which treat Lyme disease, without significantly disrupting the gut microbiome, would be an important advance. Accordingly, there is a need in the art for new compounds for, and new treatments and/or preventative measures against, diseases associated with spirochetes, such as Lyme disease.

### SUMMARY

The present disclosure provides methods related to the use of Hygromycin A. In some embodiments, the disclosure provides a method of reducing Borreliella in an animal. Such methods may include contacting non-human animal with a Hygromycin A bait. Tissue samples may be obtained from the animal and Borreliella levels may be measured in the animal. The Hygromycin A bait may include Hygromycin A at a concentration of from about 100 mg/kg to about 1000 mg/kg, of the animal. In some embodiments, contacting the animal with the Hygromycin bait may reduce the levels of Borreliella. In some embodiments, the animal may be a rodent. As a non-limiting example, the rodent may belong to Peromyscus spp. They Hygromycin A concentration in the bait may be from about 0.001 mg/g to about 5 mg/g. In some aspects, the animal may be contacted with the baits described herein for a duration from about 3 months to about 3 years. As a non-limiting example, the animal may be contacted with the Hygromycin A bait for a duration of about 6 months. Hygromycin A bait may be refreshed at least biweekly, and in some instances may be refreshed more often.

In some embodiments, the Hygromycin A baits may be used to reduce Borreliella in a tick, such as, but not limited to Ixodes spp. The Borreliella according to the present disclosure may be B. burgdorferi, B. afzelii, B. garinii, B. bavariensis or B. turcica.

Also provided herein is a bait composition. Such compositions may include Hygromycin A and a carrier. Hygromycin A may be present at a concentration of about 0.001 mg/g to about 5 mg/g.

The present disclosure also provides a method of reducing the transmission of Borreliella from an animal to a human. Such methods may include contacting the animal with a Hygromycin A bait. This in turn may reduce the Borreliella levels in the animal, thereby reducing the Borreliella transmitted to the human.

The present disclosure provides a method of increasing the sensitivity of an organism to Hygromycin A. Such methods may include, ectopically expressing bmpD (SEQ ID NO. 12) in the organism. The MIC of Hygromycin A upon ectopic expression of bmpD may be measured. In some embodiments, the sensitivity of the organism to Hygromycin A may be increased by from about 1-fold to about 10 fold. As a non-limiting example, the sensitivity may be increased by 4 fold. In some embodiments, the sensitivity may be increased by 8 fold.

### BRIEF DESCRIPTION OF THE DRAWINGS

FIG. 1 shows the measurement of protein synthesis inhibition using the GFP inducible (5 μg/ml anhydrotetracycline, ATC) B. burgdorferi strain pCRW53 and FACS. The median fluorescent intensity of 105 analyzed cells is shown.

FIG. 2 shows B. burgdorferi and E. coli cells incubated at indicated concentrations of hygromycin A for 1 min or 40 min and intracellular accumulation was quantified by UHPLC/MS. Mean±SD, N=2. Linear regression is shown in dashed lines. Slopes are significantly different between B. burgdorferi and E. coli (p<0.0001).

FIG. 3 shows the intracellular accumulation of hygromycin A in E. coli ΔtolC. Accumulation of hygromycin A was quantified in cells with the intact outer membrane and in hyper-porinated cells expressing FhuA siderophore receptor that forms a large pore in the outer membrane. Cells were incubated at indicated concentrations of hygromycin A for 40 min and intracellular accumulation was quantified by UHPLC/MS. Mean±SD, N=2.

FIG. 4 shows uptake assay. B. burgdorferi and E. coli cells were incubated at indicated concentrations of hygromycin A for 1 min and intracellular accumulation was quantified by UHPLC/MS. Mean±SD, N=2 (****=p<0.0001, ***=p<0.001, *=p<0.05).

FIG. 5 shows uptake assay. B. burgdorferi and E. coli cells were incubated at indicated concentrations of hygromycin A for 1 min and intracellular accumulation was quantified by UHPLC/MS. Mean±SD, N=2 (****=p<0.0001, ***=p<0.001, *=p<0.05).

FIG. 6 shows intracellular accumulation of hygromycin A in B. turcica and the B. turcica hygromycin A resistant mutant, KLEx1. Cells were incubated at indicated concentrations of hygromycin A for 1 min and intracellular accumulation was quantified by UHPLC/MS. Mean±SD, N=6. Note: For 16 ug/ml Hygromycin A addition, hygromycin A accumulation was below the limit of quantification.

FIG. 7A shows Hygromycin A MIC fold-change of B. burgdorferi overexpressing bmpD as compared to B. burgdorferi wild-type (upper panel), of E. coli dTolC overexpressing bmpD as compared to E. coli dTolC under repressing (M9+Glucose), inducing (M9+Arabinose) and regular LB growth conditions (middle panel) and with addition of adenosine (A, lower panel).

FIG. 7B shows Hygromycin A MIC fold-change of B. burgdorferi overexpressing bmpD as compared to B. burgdorferi wild-type or Hygromycin A resistant B. burgdorferi.

FIG. 8 shows the change in alpha diversity based on the Simpson index of the murine fecal microbiome from before to after treatment with hygromycin A (HygA) (per os), amoxicillin (Amox) (per os), or ceftriaxone (Cef) (subcutaneous). Mice were infected with B. burgdorferi N40 and treated twice a day for 5 days or were untreated (ND). Stool was collected before and after treatment and sequenced for the 16S rRNA gene and the alpha diversity was calculated using the Simpson Index metric. Each point represents the change in the Simpson index from before to after treatment for an individual mouse across three individual experiments. Bars represent the mean. Statistical significance was calculated using a one-way ANOVA with Tukey's multiple comparisons test (**=p<0.01, ***=p<0.001).

FIG. 9A and FIG. 9B show the change in relative abundance (%) of the most abundant genera in the murine gut microbiome from before to after treatment with hygromycin A (per os), amoxicillin (per os), or ceftriaxone (subcutaneous). Mice were infected with B. burgdorferi N40 and treated twice a day for 5 days or were untreated. Stool was collected before and after treatment and was sequenced for the 16S rRNA gene. For FIG. 9A, each point represents the change in the relative abundance of the respective genus for an individual mouse across three individual experiments whereas for FIG. 9B, each point represents the change in the relative abundance of the respective family for an individual mouse across three individual experiments. Bars represent the mean.

FIG. 10 shows the quantification of B. burgdorferi in tissues (ear, muscle) of animals that ingested baits. The amount of 16S rRNA was converted into cell count as described in Materials and Methods. Infected mice were given hygromycin A bait (HygA) or doxycycline bait (Doxy); infected non-treated group (ND) and non-infected group (NI) were given drug-free bait for 5 days.

### DETAILED DESCRIPTION

### I. Introduction

The incidence and geographic distribution of Lyme disease in the U.S. has increased steadily since its first description in 1977. Efforts to stem the spread of the disease through controlling the population of its tick vector and/or the mouse reservoirs of the disease have met with only limited success. The only approved human vaccine to protect against Lyme disease was removed from the market by its manufacturer, further highlighting the need for new approaches to controlling the disease.

Lyme disease is a significant public health problem in the U.S. One potential approach to the control of Lyme disease is to reduce carriage of the organisms in their wild-life reservoirs. New therapeutic strategies that do not cause the development of resistance to antibiotics used for human treatment are desirable. In addition, there is a need to identify therapeutic agents that can be administered to mice and ticks that serve as the reservoir for Lyme disease causing bacteria such as, Borrelia. Decreasing the carriage of the Lyme disease causing bacteria in the wildlife reservoirs and vectors, may reduce the incidence of human disease.

Treatment of mice with an antibiotic, doxycycline, has been shown to be highly effective in eradicating Borrelia burgdorferi from its reservoir hosts. However, there is legitimate concern for development of resistance, both in B. burgdorferi and in other organisms that may be exposed to the antibiotic should it be widely distributed. Doxycycline is an important antibiotic in the treatment of multiple different human infections and in some cases such as Anaplasma or Rocky Mountain Spotted Fever, the only approved agent available.

The present disclosure provides therapeutic agents targeted towards the mouse and tick reservoirs of the disease. Provided herein are methods of using compounds such as, but not limited to, HygA as an environmental antibiotic. This methods and compositions described herein may control the organism in its major reservoirs.

Despite increased awareness of Lyme disease, the incidence of human infections with Borrelia burgdorferi has risen steadily over the last 15 years. While Lyme disease was originally localized to a few highly endemic regions in the United States, the geographic range of Ixodes ticks infected with B. burgdorferi has been expanding rapidly. Measures aimed at controlling human exposures to the infected tick vector, such as acaricides and repellants have not had a large impact on the spread of the disease. Strategies to control Lyme disease by reductions in animal populations such as deer removal and habitat modification have been shown to be effective in isolated situations but are not implementable on a large scale for political and practical reasons. As a result, new strategies for controlling infection in the wild reservoirs of Lyme disease need to be explored. Two major approaches have been tried for directly controlling infection in wild reservoirs. Reservoir-targeted vaccination of mice with outer surface protein A recombinant protein has undergone field trials and showed modest efficacy in reducing carriage of the organism in mice and ticks. In contrast, field trials of antibiotic treatment of animals using doxycycline laden baits have shown high levels of infection clearance. However, a strategy of wide distribution of doxycycline for target animals has limited appeal due to concerns over the development of resistance, not only for B. burgdorferi, but for other human pathogens including Anaplasma phagocytophilium where doxycycline is the only approved antibiotic.

Spraying of acaricides to reduce numbers of the Ixodes tick vector for B. burgdorferi has been shown to be effective in reducing ticks but not human Lyme disease. Widespread acaricidal applications have also not gained general acceptance due to reluctance by most communities to distribute poisons of even low toxicity and for their impact on non-target insects. More targeted approaches have taken advantage of the feeding and nesting behaviors of deer or mice. While deer are not important in the lifecycle of B. burgdorferi as they are not competent hosts, deer are important hosts for adult ticks. A “four-poster” device, in which deer are coated with an acaricide as they feed, has been shown to reduce the number of ticks that are carried but has seen limited distribution due to concerns for exacerbating deer wasting disease. For control of ticks on mice, cardboard tubes containing cotton treated with permethrin (now marketed as Damminix) were distributed in zoonotic sites with the expectation that mice would line their nests with a material that would kill all ectoparasites on their fur. Results with these “tick tubes” have been mixed, with successful reduction of tick infestation at some sites but no decrease in B. burgdorferi carriage at others. Although there are multiple reservoirs for B. burgdorferi including birds and small rodents such as mice, voles and chipmunks, targeting of the main reservoir, mice, can have significant impacts on reductions of infected ticks. Models of the impact of clearing infections in mice have also concluded that it is possible to significantly reduce or, over time, even eradicate B. burgdorferi infection even without targeting other reservoir hosts. Reservoir targeted vaccination to reduce carriage of B. burgdorferi in its murine hosts has been attempted.

Hand capture and vaccination of mice by injection resulted in a 19-25% reduction in infected nymphal ticks the season following vaccination. The use of an oral vaccine consisting of recombinant outer surface protein A (OspA) has shown some efficacy both in the laboratory and in field trials. However, immunogenicity of this vaccine is weak and field trials showed very variable results. There is reluctance to release a genetically altered infectious virus and the vaccine faces significant regulatory hurdles.

Previous studies have shown that distribution of baits containing doxycycline to mice are highly effective in reducing carriage of B. burgdorferi and A. phagocytophilium both in mice and ticks, providing a strong conceptual rationale for our project. After 2 years of application, doxycycline reduced recovery of B. burgdorferi in mammals by 87% and in ticks by 94%. A second trial combining doxycycline with an acaricide, fipronil in boxes also showed excellent efficacy. However, as doxycycline is a first line antibiotic for treatment of human Lyme disease as well as the only recommended antibiotic to treat A. phagocytophilium, there has been little enthusiasm for this strategy due to concerns over the development of resistance. Compared with interventions aimed at humans such as vaccination, which require continued investment and do nothing to stem the expansion of the disease to new areas, reservoir targeted approaches have the potential for long term reductions in infections, prevention of expansion and even eradication of the disease in its endemic hosts entirely. The effectiveness of reservoir targeted disease control also benefits from the “transmission threshold”, which is the number of animals/ticks that need to be cleared of infection in order to stop transmission in the population. The transmission threshold does not require reducing infection rates to zero. Even modest decreases in the infection rate may be sufficient to get below the transmission threshold and potentially eradicate Lyme disease in areas over time. Development of novel antibiotics for use in wildlife treatment that do not result in cross resistance with human antibiotics would be an important tool for use in an integrated strategy for reduction of human Lyme disease.

Successful development of a novel antibiotic to treat B. burgdorferi infection with the goal of using it in reservoir animals would represent an advantageous strategy against Lyme disease. The present disclosure provides animal antibiotics that decrease carriage of B. burgdorferi in mice and ticks but do not result in cross-resistance of human pathogens to human antibiotics. Our identification of HygA as such a compound allows testing of a reservoir targeted treatment strategy.

### II. Compositions

In some embodiments, the compositions of the disclosure may include any of the compounds or formula described in International Patent Publication WO2020041179, the contents of which are herein incorporated by reference in its entirety. In some embodiments, the compositions of the disclosure may include a compound having the structure according to Formula I of the International Patent Publication WO2020041179.

In some embodiments, the compound has activity against B. burgdorferi.

In some embodiments the compound may be an antimicrobial produced by Streptomyces hygroscopicus. As a non-limiting example, the compound may be Hygromycin A. Hygromycin A targets the ribosomes and is efficiently taken up by B. burgdorferi.

In some embodiments, the present disclosure provides a compound described herein, or a salt, hydrate or solvate thereof, or a combination thereof. In some embodiments, the disclosure provides a compound described herein, or a salt thereof. In some embodiments, the salt is a pharmaceutically acceptable salt. In some embodiments, the disclosure provides a compound described herein, or a hydrate thereof. In some embodiments, the disclosure provides a compound described herein, or a solvate thereof. In some embodiments, the disclosure provides a salt of a compound described herein. In some embodiments, the disclosure provides a pharmaceutically acceptable salt of a compound described herein. In some embodiments, the disclosure provides a hydrate of a compound described herein. In some embodiments, the disclosure provides a solvate of a compound described herein.

### Bait

The present disclosure provides a bait composition. As used herein, a bait may refer to composition containing an active compound and a carrier that may be palatable or at least partially edible to a target animal and which may be used to lure the target animal to a particular location/position and/or to consume the active compound in the bait.

In some embodiments, the bait compositions may include Hygromycin A as the active compound and may herein be referred to as a Hygromycin A bait. In some embodiments, the compound of the disclosure can be formulated at therapeutic concentrations in standard size mouse food pellets.

In some embodiments, the carrier may be Bait Formula or a nutrient mouse chow. In some embodiments, the compound of the disclosure is water soluble and is directly mixed with the feed prior to forming pellets. In some embodiments, the bait compositions can be deployed by scattering in environmental settings with high populations of rodents or placed within T-shaped bait-stations built of PVC piping that allows feeding by mice but blocks access by humans. Baits can be formulated using Bait Formula supplied by FoodSource or a similar nutrient mouse chow base.

In some embodiments, the carrier may include bait formula, and/or peanut butter. As a non-limiting example, bait compositions may include 2 g of bait formula, mixed with 0.5 g of peanut butter and 2 mL of boiling water. After mixing and cooling, the compound may be added and stirred together with a sterile spatula. The bait may be shaped into a square and allowed to air dry at room temperature. Miniature versions may be prepared in wells of a 96-well plate with standard 200 pL working volume. Food coloring may also be added to make the bait more visible to mice (Bhattacharya et al Vaccine. 2011 Oct. 13; 29(44): 7818-7825; herein incorporated by reference). In some embodiments, any of the bait compositions described in U.S. Pat. No. 9,078,924 may be useful in the present disclosure (the contents of which are herein incorporated by reference in its entirety).

A Hygromycin A bait may be prepared by mixing crushed mouse chow and peanut butter at an equal volume ratio. Compounds such as hygromycin A may be added at a final concentration in the range of 0.001 to 5 mg/g of bait.

In some embodiments, the bait compositions of the disclosure may include about 100-1000 mg/kg of HygA. In some embodiments, the bait compositions of the disclosure may include about 50 mg/kg of HygA. In some embodiments, the bait compositions of the disclosure may include about 200 mg/kg of HygA. In some embodiments, the bait compositions may contain nuts, grains and a natural gum material.

### III. Methods of Use

### Reducing Borreliella Levels in an Animal

The present disclosure provides methods related to the use of Hygromycin A. In some embodiments, the disclosure provides a method of reducing Borreliella in an animal. Such methods may include contacting non-human animal with a Hygromycin A bait. Tissue samples may be obtained from the animal and Borreliella levels may be measured in the animal. The Hygromycin A bait may include Hygromycin A at a concentration of from about 100 mg/kg to about 1000 mg/kg, of the animal. As a non-limiting example, the concentration of Borreliella may be about 100 mg/kg. In another example, the concentration of Borreliella may be about 200 mg/kg.

In some embodiments, contacting the animal with the Hygromycin bait may reduce the levels of Borreliella. In some embodiments, the animal may be a rodent. As a non-limiting example, the rodent may belong to Peromyscus spp. The Hygromycin A concentration in the bait may be from about 0.001 mg/g to about 5 mg/g. In some aspects, the animal may be contacted with the baits described herein for a duration from about 3 months to about 3 years. As a non-limiting example, the animal may be contacted with the Hygromycin A bait for a duration of about 6 months. As a non-limiting example, the animal may be contacted with the Hygromycin A bait for a duration of about 1 year. As a non-limiting example, the animal may be contacted with the Hygromycin A bait for a duration of about 2 years. Hygromycin A bait may be refreshed at least biweekly, and in some instances may be refreshed more often, e.g., daily or weekly.

In some embodiments, the Hygromycin A baits may be used to reduce Borreliella in a tick, such as, but not limited to Ixodes spp. The Borreliella according to the present disclosure may be B. burgdorferi, B. afzelii, B. garinii, B. bavariensis or B. turcica. The reduction in the Borreliella levels in the animal may be 50%, 60%, 70%, 80%, 90%, 100%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, and/or 90-100%.

### Reducing Transmission of Borreliella

The present disclosure also provide a method of reducing the transmission of Borreliella from animals to humans. Such methods may include contacting an animal with a Hygromycin A bait. This in turn may reduce the Borreliella levels in the animal, thereby reducing the Borreliella transmitted to humans.

The Hygromycin A bait may include Hygromycin A at a concentration of from about 100 mg/kg to about 1000 mg/kg, of the animal. As a non-limiting example, the concentration of Borreliella may be about 100 mg/kg. In another example, the concentration of Borreliella may be about 200 mg/kg.

In some embodiments, the animal may be a rodent. As a non-limiting example, the rodent may belong to Peromyscus spp. The Hygromycin A concentration in the bait may be from about 0.001 mg/g to about 5 mg/g. In some aspects, the animal may be contacted with the baits described herein for a duration from about 3 months to about 3 years. As a non-limiting example, the animal may be contacted with the Hygromycin A bait for a duration of about 6 months. As a non-limiting example, the animal may be contacted with the Hygromycin A bait for a duration of about 1 year. As a non-limiting example, the animal may be contacted with the Hygromycin A bait for a duration of about 2 years. Hygromycin A bait may be refreshed at least biweekly, and in some instances may be refreshed more often, e.g., daily or weekly. The Borreliella according to the present disclosure may be B. burgdorferi, B. afzelii, B. garinii, B. bavariensis or B. turcica. The reduction in the transmission of Borreliella levels in the animal may be 50%, 60%, 70%, 80%, 90%, 100%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, and/or 90-100%.

### Tuning Sensitivity to Borreliella

The present disclosure provides a method of increasing the sensitivity of an organism to Hygromycin A. Such methods may include, ectopically expressing proteins such as, but not limited to, bmpD (UniProt ID: POCL55; SEQ ID NO: 12), a periplasmic substrate-binding protein of an ABC-type purine nucleoside transporter; BB0678 (UniProt ID: 051621; SEQ ID NO: 13); BB0679 (UniProt ID: 051622; SEQ ID NO: 14) and/or the ATP-binding protein BB0677 (subsequently referred to as BmpDEFG; UniProt ID: 051620; SEQ ID NO: 15).

The MIC of Hygromycin A upon ectopic expression of bmpD may be measured to determine the sensitivity to HygA. In some embodiments, the sensitivity of the organism to Hygromycin A may be increased by from about 1-fold to about 10 fold, from about 10 fold to about 100 fold, or from about 100 fold to about 1000 fold. As a non-limiting example, the sensitivity may be increased by 4 fold. In some embodiments, the sensitivity may be increased by 8 fold.

### Inhibiting Spirochete Growth or Killing a Spirochete

The compounds of the disclosure exhibit potency against spirochetes, and therefore have the potential to treat, and/or prevent a spirochete infection, or kill and/or inhibit the growth of a spirochete.

In another aspect, the spirochete is inside, or on the surface of an animal. In another exemplary embodiment, the animal is described herein. In another exemplary embodiment, the animal is a human.

In an exemplary embodiment, the spirochete infection is treated and/or prevented, or the spirochete is killed or its growth is inhibited, through oral administration of the compound of the disclosure. In an exemplary embodiment, the spirochete infection is treated and/or prevented, or the spirochete is killed or its growth is inhibited through intravenous administration of the compound of the disclosure.

In an exemplary embodiment, the spirochete is selected from the group consisting of the Leptospirales order, the Brachyspirales order, the Brevinematales order, and the Spirochaetales order. In an exemplary embodiment, the spirochete is selected from the group consisting of the Leptospiraceae family, the Brachyspiraceae family, the Brevinemataceae family, the Borrehaceae family, and the Spirochaetaceae family. In an exemplary embodiment, the spirochete is selected from the group consisting of the Leptospiraceae family, the Brachyspiraceae family, the Brevinemataceae family, the Borrehaceae family, and the Spirochaetaceae family. In an exemplary embodiment, the spirochete is selected from the group consisting of the Leptonema genus, the Leptospira genus, the Turneriella genus, the Brachyspira genus, the Brevinema genus, the Exihspira genus, the Borreliella genus, the Borrelia genus, the Cristispira pectinis genus, the Clevelandina reticulitermitidis genus, the Diplocalyx calotermitidis genus, the Hollandina pterotermitidis genus, the Pillotina calotermitidis genus, the Spironema culicis genus, the Spirochaeta genus, and the Treponema genus. In an exemplary embodiment, the spirochete is of the Treponema genus. In an exemplary embodiment, the spirochete is of the Leptospira genus. In an exemplary embodiment, the spirochete is of the Borrelia genus. In an exemplary embodiment, the spirochete is of the Brachyspira genus.

In an exemplary embodiment, the spirochete is Borrelia burgdorferi. In an exemplary embodiment, the spirochete is Borrelia mayonii. In an exemplary embodiment, the spirochete is Borrelia afzelii. In an exemplary embodiment, the spirochete is Borrelia garinii. In an exemplary embodiment, the spirochete is Borrelia recurrentis. In an exemplary embodiment, the spirochete is Treponema pallidum. In an exemplary embodiment, the spirochete is Brachyspira pilosicoh. In an exemplary embodiment, the spirochete is Brachyspira aalborgi.

### Spirochete Infection

The compounds of the disclosure exhibit potency against spirochete, and therefore have the potential to be used to treat and/or prevent a spirochete infection.

In a further aspect, the disclosure provides a method of treating and/or preventing a spirochete infection, or a method of killing and/or inhibiting the growth of a spirochete, said method comprising: contacting said spirochete with an effective amount of a compound of the disclosure, thereby killing and/or inhibiting the growth of the spirochete.

In a further aspect, the disclosure provides a method of treating a spirochete infection comprising administering to an animal suffering from the infection an effective amount of a compound of the disclosure, or a pharmaceutically acceptable salt thereof, thereby treating the spirochete infection.

In a further aspect, the disclosure provides a method of preventing a spirochete infection comprising administering to an animal a prophylactic amount of a compound of the disclosure, or a pharmaceutically acceptable salt thereof, thereby preventing the spirochete infection.

### Diseases

The compounds of the disclosure exhibit potency against spirochetes, and therefore have the potential to achieve therapeutic efficacy in the animals described herein. In another aspect, the disclosure provides a method of treating and/or preventing a disease. In an exemplary embodiment, the method includes administering to the animal a therapeutically effective amount of a compound of the disclosure, thereby treating and/or preventing the disease. In an exemplary embodiment, the compound of the disclosure can be used in human or veterinary medical therapy, particularly in the treatment or prophylaxis of spirochete-associated disease. In an exemplary embodiment, the compound is described herein, or a salt, hydrate or solvate thereof, or a combination thereof. In an exemplary embodiment, the disclosure provides a compound described herein. In an exemplary embodiment, the disclosure provides a compound described herein, or a salt, hydrate or solvate thereof. In an exemplary embodiment, the disclosure provides a compound described herein, or a salt thereof. In another exemplary embodiment, the compound of the disclosure is a compound described herein, or a pharmaceutically acceptable salt thereof. In an exemplary embodiment, the compound is a compound described herein, or a pharmaceutically acceptable salt thereof. In an exemplary embodiment, the compound is according to a formula described herein, or a pharmaceutically acceptable salt thereof. In an exemplary embodiment, the compound is part of a combination described herein. In an exemplary embodiment, the compound is part of a pharmaceutical formulation described herein. In another exemplary embodiment, the disease is a systemic disease. In another exemplary embodiment, the disease is a topical disease. In an exemplary embodiment, the animal being administered the compound is not otherwise in need of treatment with the compound. In an exemplary embodiment, the disease is treated through oral administration of a compound of the disclosure and/or a combination of the disclosure. In an exemplary embodiment, the disease is treated through intravenous administration of a compound of the disclosure and/or a combination of the disclosure. In an exemplary embodiment, the disease is treated through subcutaneous administration of a compound of the disclosure and/or a combination of the disclosure. In another exemplary embodiment, the disease is associated with a spirochete described herein. In an exemplary embodiment, the disease is associated with a Treponema species. In an exemplary embodiment, the disease is associated with a Leptospira species. In an exemplary embodiment, the disease is associated with a Borrelia species. In an exemplary embodiment, the disease is associated with a Brachyspira species. In another exemplary embodiment, the disease is leptospirosis. In another exemplary embodiment, the disease is Lyme disease. In another exemplary embodiment, the disease is relapsing fever. In another exemplary embodiment, the disease is syphilis. In another exemplary embodiment, the disease is yaws. In another exemplary embodiment, the disease is intestinal spirochetosis. In another exemplary embodiment, the disease is gingivitis or periodontitis.

In an exemplary embodiment, for any of the methods described herein, the animal is selected from the group consisting of human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, for any of the methods described herein, the animal is selected from the group consisting of a human, cattle, goat, pig, sheep, horse, cow, bull, dog, guinea pig, gerbil, rabbit, cat, chicken and turkey. In another exemplary embodiment, for any of the methods described herein, the animal is a human.

In an exemplary embodiment, for any of the methods described herein, a compound of the disclosure, a compound described herein or a pharmaceutically acceptable salt thereof, and/or a pharmaceutical formulation described herein can be used.

### IV. Dosing and Administration

In some embodiments, the methods of the disclosure can be employed through the topical application of the compounds described herein. Topical administration includes for example, transmucosal, transdermal, ungual and transungual routes of administration. The topical compositions useful in the subject disclosure can be made into a wide variety of product types. These include, but are not limited to, lotions, creams, gels, sticks, sprays, ointments, pastes, foams, mousses, masks, eye ointments, eye or ear drops, impregnated dressings, wipes, cleansers including soaps, body washes and shampoos, and make-up products, such as bases, blushes, lipsticks, and eye shadows, among others. These product types can comprise several types of carrier systems including, but not limited to particles, nanoparticles, and liposomes. If desired, disintegrating agents can be added, such as the cross-linked polyvinyl pyrrolidone, agar or alginic acid or a salt thereof such as sodium alginate. Techniques for formulation and administration can be found in Remington: The Science and Practice of Pharmacy supra. The formulation can be selected to maximize delivery to a desired target site in the body. The formulations can also include various conventional colorants, fragrances, thickeners, preservatives, humectants, emollients, demulcents, solubilizing excipients, dispersants, penetration enhancers, plasticizing agents, preservatives, stabilizers, demulsifiers, wetting agents, sunscreens, emulsifiers, moisturizers, astringents, deodorants, and the like, which can be added to provide additional benefits such as, for example, improving the feel and/or appearance of the topical preparation.

The compositions disclosed herein may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Alternatively, the compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For administration to non-human animals, the composition containing the therapeutic compound may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also be added as a food or drink supplement for humans.

Dosage levels on the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a single dosage form will vary depending upon the condition being treated and the particular mode of administration. Dosage unit forms will generally contain between from about 1 mg to about 500 mg of an active ingredient.

Frequency of dosage may also vary depending on the compound used and the particular disease treated. However, for treatment of most disorders, a dosage regimen of 4 times daily or less is utilized. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

Preferred compounds of the disclosure will have desirable pharmacological properties that include, but are not limited to, oral bioavailability, low toxicity, low serum protein binding and desirable in vitro and in vivo half-lives. Penetration of the blood brain barrier for compounds used to treat CNS disorders is necessary, while low brain levels of compounds used to treat peripheral disorders are often utilized.

The amount of the composition required for use in treatment will vary not only with the particular compound selected but also with the route of administration, the nature of the condition being treated and the age and condition of the patient and will ultimately be at the discretion of the attendant physician or clinician.

In some embodiments, the pharmaceutical composition described herein includes an additional active ingredient. In another exemplary embodiment, the additional active ingredient is a compound that has been approved for human use by the United States Food and Drug Administration.

### V. Definitions

As used herein, the singular forms “a”, “an”, and “the” include plural references unless the context clearly dictates otherwise. For example, reference to “an active agent” includes a single active agent as well as two or more different active agents in combination. It is to be understood that present teaching is not limited to the specific dosage forms, carriers, or the like, disclosed herein and as such may vary.

The abbreviations used herein generally have their conventional meaning within the chemical and biological arts.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy. 21st Ed., Lippincott, Williams & Wilkins (2005) which is incorporated herein by reference.

By “effective” amount of a drug, formulation, or permeant is meant a sufficient amount of an active agent to provide the desired local or systemic effect. A “Topically effective,” “pharmaceutically effective,” or “therapeutically effective” amount refers to the amount of drug needed to effect the desired therapeutic result.

The term “pharmaceutically acceptable salt” is meant to include a salt of a compound of the disclosure, which is prepared with relatively nontoxic acids or bases, depending on the particular substituents found on the compounds described herein. When compounds of the disclosure contain relatively acidic functionalities, base addition salts can be obtained by contacting the neutral form of such compounds with a sufficient amount of the desired base, either neat or in a suitable inert solvent

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The term “excipients” is conventionally known to mean carriers, diluents and/or vehicles used in formulating drug compositions effective for the desired use.

The terms “effective amount” or a “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

The phrases “active ingredient”, “therapeutic agent”, “active”, or “active agent” mean a chemical entity which can be effective in treating a targeted disorder, disease or condition.

The phrase “pharmaceutically acceptable” means moieties or compounds that are, within the scope of medical judgment, suitable for use in humans without causing undesirable biological effects such as undue toxicity, irritation, allergic response, and the like, for example.

The phrase “oral dosage form” means any pharmaceutical formulation administered to a subject via the oral cavity. Exemplary oral dosage forms include tablets, capsules, films, powders, sachets, granules, solutions, solids, suspensions or as more than one distinct unit (e.g., granules, tablets, and/or capsules containing different actives) packaged together for co-administration, and other formulations known in the art. An oral dosage form can be one, two, three, four, five or six units. When the oral dosage form has multiple units, all of the units are contained within a single package, (e.g. a bottle or other form of packaging such as a blister pack). When the oral dosage form is a single unit, it may or may not be in a single package. In a preferred embodiment, the oral dosage form is one, two or three units. In a particularly preferred embodiment, the oral dosage form is one unit.

The phrase “unit”, as used herein, refers to the number of discrete objects to be administered which comprise the dosage form. In some embodiments, the dosage form includes a compound of the disclosure in one capsule. This is a single unit. In some embodiments, the dosage form includes a compound of the disclosure as part of a therapeutically effective dosage of a cream or ointment. This is also a single unit.

In some embodiments, the dosage form includes a compound of the disclosure and another active ingredient contained within one capsule, or as part of a therapeutically effective dosage of a cream or ointment. This is a single unit, whether or not the interior of the capsule includes multiple discrete granules of the active ingredient. In some embodiments, the dosage form includes a compound of the disclosure in one capsule, and the active ingredient in a second capsule. This is a two unit dosage form, such as two capsules or tablets, and so such units are contained in a single package. Thus the term ‘unit’ refers to the object, which is administered to the animal, not to the interior components of the object.

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” is used herein to refer to the partial or full blockade of the growth of a spirochete described herein.

Embodiments of the disclosure also encompass compounds that are poly- or multi-valent species, including, for example, species such as dimers, trimers, tetramers and higher homologs of the compounds of use in the disclosure or reactive analogues thereof.

The details of one or more embodiments of the disclosure are set forth in the accompanying description below. Although any materials and methods similar or equivalent to those described herein can be used in the practice or testing of the present disclosure, the materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Dosing and Safety Evaluation of Hygromycin a in Mice

The activity of HygA against B. burgdorferi has been previously described in International Patent Publication WO2020041179A1 (the contents of which are herein incorporated by reference in its entirety). H.7gA was discovered in 1953 from a strain of Streptomyces hygroscopicus isolated by Selman Waksman. Since HygA did not show strong activity against any test pathogen, it was not pursued as a human antibiotic. 30 years later, a group from Japan determined that the compound had good activity against a spirochetal organism, Brachyspira (formerly Treponema) hyodysenteriae, and showed efficacy in treating pig dysentery caused by this pathogen. Preliminary testing has shown that the biosafety window of Hygromycin A is >500 mg/kg, which is consistent with previous reports. HygA is structurally different than Hygromycin B, which is also derived from Streptomyces. HygB is an aminoglycoside antibiotic that has been used extensively in the past in animal feed and has much broader spectrum of activity than HygA.

To screen for natural products against B. burgdorferi, soil bacteria were grown in modified seed broth (MSB). After 2-7 days, culture supernatants were collected, dried and resuspended in water to obtain a 15× concentrated extract. A screen was then performed by adding the extract to wells of microtiter plates containing B. burgdorferi, at a final concentration of 0.15× (as compared to the initial supernatant). A screen of 549 extracts produced a hit rate of 6% against B. burgdorferi. A counter screen against S. aureus and E. coli resulted in 15 candidates selective for B. burgdorferi, (2.7% selective hit rate).

One of the hits that showed potent activity against B. burgdorferi and good selectivity, was from Streptomyces hygroscopicus, a common species of Streptomyces. An extract of S. hygroscopicus was separated into 40 fractions using high-performance liquid chromatography (HPLC). Bioassay-driven purification was performed to identify the active compound. Crude extraction utilizing XAD-16N polymer resin from culture supernatant resulted in removing majority of contaminants and captured the active compound. The eluate was subject to RP-HPLC purification by peak collection. Each peak was tested for inhibition of B. burgdorferi, and the peak corresponding to the active compound was identified. Mass spectrum analysis of the active compound give a molecular ion peak at m/z 512.2 (M+H) which matches the mass of Hygromycin A. Fragmentation characterization by tandem mass spectrometry establishes the partial connectivity of the amide hygromycin A moieties. NMR analysis confirms HygA structurally by the 13C and 1H NMR spectral values consistent with reported values. Mass spectrum analysis, fragmentation characterization by tandem mass spectrometry and NMR elucidates the active compound as HygA.

The spectrum of activity of hygromycin A against a panel of representative organisms was analyzed. Hygromycin A was found to be highly active against spirochetes (Table 1). Activity of compounds acting against B. burgdorferi was determined by broth microdilution under microaerophilic conditions. HygA was equally active against B. burgdorferi and the species responsible for the disease in Europe—B. afzelii, B. garinii and B. bavariensis (MIC 0.25 μg/ml). The highest activity for HygA was against Treponema pallidum, the causative agent of syphilis, with an MIC of 0.03 μg/ml (determined by monitoring spirochetes in a co-culture with rabbit cells). HygA was also quite active against environmental spirochetes such as Alkalispirochaeta americana. Activity against S. aureus which was used in the counter-screen is 64 times lower compared to B. burgdorferi. HygA is fairly ineffective against both gram positive and negative gut symbionts representing the major taxonomic groups (Table 1).

TABLE 1
Bacterial sensitivities to HygA

| | |
|---|---|
| Organism and genotype | MIC (ug) |

Spirochete

| | |
|---|---|
| Treponema pallidum | 0.03 |
| Borrelia burgdorferi BbP1286 | 0.12 |
| Borrelia burgdorferi (B31, N40, 297) | 0.25 |
| Borrelia afzelii | 0.25 |
| Borrelia garinii | 0.25 |
| Borrelia bavariensis | 0.25 |
| Borrelia turcica | 0.5 |
| Alkalispirochaeta americana | 0.5 |
| Leptospira biflexa | 4 |

Other bacteria

| | |
|---|---|
| Staphylococcus aureus | 8 |
| Escherichia coli WO153 | 16 |
| Shigella sonnei | 64 |
| Escherichia coli MG1655 | 128 |
| Pseudomonas aeruginosa | >128 |
| Salmonella typhimurium | >128 |
| Enterobacterium | >128 |

Symbiotic gut bacteria

| | |
|---|---|
| Bifidobacterium longum | 8 |
| Lactobacillus reuteri | 32 |
| Bacteroides fragilis | >128 |
| Enterococcus faecalis | >128 |
| Escherichia coli Nissle | >128 |

### Example 2: Evaluation of Cytotoxicity of Hygromycin A

A microplate Alamar Blue assay (MABA/resazurin) was used to determine the cytotoxicity of Hygromycin A. The cell lines used were FaDu pharynx squamous cell carcinoma (ATCC HTB-43), HepG2 liver hepatocellular carcinoma (ATCC HB-8065), and HEK293-RFP human embryonic kidney red fluorescent protein tagged (GenTarget SC007) cells, all cultured in Eagle's Minimum Essential Medium supplemented with 10% fetal bovine serum. Exponentially growing cells were seeded into a 96-well, flat bottom, tissue culture treated plate (Corning) and incubated at 37° C. with 5% CO2. After 24 hours, the medium was aspirated and replaced with fresh medium containing HygA (concentrations from 1 to 512 μg/ml). After 72 hours of incubation at 37° C. with 5% CO2, resazurin (Acres Organics) was added to each well to a final concentration of 0.15 mM. After three hours, the As44 and Asgo were measured using a BioTek Synergy H 1 microplate reader to determine cell viability. Experiments were performed with biological replicates. No cytotoxicity was seen in any cell line up to the maximal concentration tested, 51211 g/ml.

### Example 3: Efficacy of HygA in Treatment of B. burgdorferi Infected Mice

Mice were infected with B. burgdorferi and allowed the infection to establish for 3 weeks. Infection was confirmed by culturing an ear punch biopsy. HygA was then administered to mice at 75 mg/kg twice per day intraperitoneally for 5 days. Mice were then sacrificed, and their tissues cultured and processed for PCR using 16S primers for B. burgdorferi. 0/4 mice treated with HygA vs 4/4 control treated mice had positive cultures or PCRs for B. burgdorferi. The oral administration of HygA was also tested. Mice were infected with B. burgdorferi. Groups of 4 mice were gavaged with HygA twice per day (Table 2). B. burgdorferi were cultured from mice administered with 10 and 25 mg/kg, but not at any of the higher doses. PCRs were similarly negative from animals from ear, joint and heart tissue for mice at doses above 50 mg/kg. HygA was also administered to mice in baits. A bait formulation from Foodsource Lures Corp was utilized for these experiments. The animals were given bait once a day for 5 days containing HygA (200 mg/kg) or doxycycline (100 mg/kg). Control groups included uninfected and infected animals, both given bait alone. Animals were sacrificed 3 days after completion of antibiotic treatment and tissues were processed for culture and PCR (Table 2). All animals treated with either HygA or doxycycline were found to be clear of infection by culture and PCR. Importantly, there were no signs of toxicity at any of the doses used.

TABLE 2
Efficacy of hygromycin A for treatment of B. burgdorferi infected mice

| | | | | Mean # of | B. burgdorferi |
|---|---|---|---|---|---|
| | | | | B. burgdorferi/10 mg of | culture (% |
| Compositions | Dose | | Route | skin tissue (qPCR) | positive) |
| Saline | | | IP injection | 700 | 100 |
| Ceftriaxone | 156 | mg/kg | IP injection | <1 | 0 |
| Hygromycin A | 75 | mg/kg | IP injection | <1 | 0 |
| Hygromycin A | 250 | mg/kg | Oral gavage | <1 | 0 |
| Hygromycin A | 70 | mg/kg | Oral gavage | <1 | 0 |
| Hygromycin A | 50 | mg/kg | Oral gavage | <1 | 0 |
| Hygromycin A | 25 | mg/kg | Oral gavage | <1 | 50 |
| Hygromycin A | 200 | mg/kg | Bait | <1 | 0 |
| Doxycycline | 100 | mg/kg | Bait | <1 | 0 |

### Example 4: Impact of HygA on Mouse Gut Microbiomes

The microbiomes of mice given HygA (50 mg/kg oral), amoxicillin (100 mg/kg oral) or ceftriaxone (156 mg/kg subcutaneous) for 5 days. Stool samples were collected and V4 region of the 16s rRNA gene was amplified using primers and sequenced (IonTorrent). Ceftriaxone and oral amoxicillin lead to Enterococcaceae blooms, up to 98% relative abundance, in 5/5 and 4/5 mice respectively; all other commensal taxa such as Bacteroidaceae and Lactobacillaceae were depleted in these mice. In contrast, oral hygromycin treatment did not lead to increases in Enterococcaceae or Enterobacteriaceae. Bacteroidaceae decreased in 3/5 mice treated with oral hygromycin, but 1/5 mice maintained Bacteroidaceae levels and 1/5 mice had increased Bacteroidaceae, and the overall fluctuation of Bacteroidaceae (−8.5% relative abundance) was not significantly different from the untreated group. This suggests that HygA is less active against the gut microflora of mice.

### Example 5: Pharmacokinetic Properties of HygA

Single-dose PK and tolerability studies are performed in Peromyscus mice since these species are the target population. 6-8 week-old Peromyscus leucopus fusus mice that derive from outbred mice captured on Martha's Vineyard, MA or purchased from the Peromyscus Genetic Stock Center (South Carolina). The mice are maintained under barrier housing conditions and determined to be free of B. burgdorferi infection. For all animal experiments, male and female Peromyscus mice are used in equal numbers per group. HygA is delivered by oral gavage at 100 mg/kg and blood is drawn by retro-orbital bleed from four mice per time point, at 5 and 15 min; 1, 2, 4, 8 and 24 hours. No toxicity is expected at these doses. Blood is stored on ice and within 30 minutes plasma is isolated by centrifugation and stored at −80° C. An LC/MS/MS protocol (Agilent 1260 coupled to Agilent 6460 TripleQuad) for HygA using a calibration curve of the compound spiked into plasma is used to determine plasma Hygromycin A concentrations.

### Example 6: Determination of In Vivo Toxicity

Preliminary data for toxicity in C3H mice suggests the absence of toxicity in the maximal dose that was tested (500 mg/kg). This is 10-fold above the minimal efficacious dose. Structured toxicity dose testing is performed using Peromyscus mice over a wide range of dosing (100-1,000 mg/kg). Mice are observed hourly for the first 10 hours post dose and at 24 h. Information on No Acute Effect Level (NoAEL) is coupled with the PK study. The effects of longer-term administration of Hyg are examined by administering the drug to Peromyscus mice daily by bait at three different dosages for up to 16 weeks. The experiment is stopped if mice develop signs of toxicity from the cumulative doses. Because the timing of tick feeding in the wild is typically greatest during specific windows of time, if toxicity is observed with prolonged use, the baits are made available only during peak intensity periods for larval and nymphal tick feeding. Mice are observed for signs of toxicity daily.

### Example 7: Assessment of Stability of HygA Under Various Environmental Conditions

The experiment evaluates the stability of HygA in baits exposed to conditions that mice are likely encounter when deployed in the wild. Baits containing HygA are exposed to heat (>37° C.) or freezing (<0° C. over multiple freeze-thaw cycles) for varying lengths of time.

Baits are solubilized and the activity of HygA extracted from the baits is compared in vitro between compound from baits exposed to extreme temperatures or not. Longitudinal analysis of stability of HygA at room temperature (23° C.) for prolonged periods of time by weekly sampling over 6 months is also performed.

### Example 8: Testing of Efficacy in Captive Peromyscus Mice

The efficacy of HygA in Peromyscus mice infected naturally through tick infection is tested. Briefly, Peromyscus mice are infected with B. burgdorferi N40 by feeding of infected nymphal Ixodes scapularis ticks. The infection is allowed to establish for 3 weeks, and then animals will be dosed twice per day for 5 days with saline (oral gavage), ceftriaxone (156 mg/kg, subcutaneous injection), or HygA at doses calculated from the PK studies and toxicity studies (the highest NoAEL dose). Ear punch samples are taken each of the 5 days and cultured for B. burgdorferi.

Animals are sacrificed 2 days after completion of antibiotic treatment and tissue from the injection site, heart, ankle and bladder is harvested for B. burgdorferi culture and quantitative PCR performed. Again, male and female mice are used in equal numbers per group and analyzed separately and together. Animal efficacy and toxicity testing using baits is also performed. Peromyscus mice are again infected by feeding of B. burgdorferi infected nymphal ticks. The baits containing HygA, doxycycline or placebo using nuts, grains and a natural gum material to hold the mixture and the antimicrobial compound together (Foodsource). Baits containing the compound or control are placed into cages containing a family of 6-10 mice. Baited HygA is placed along with natural foods and rodent chow and the mice are allowed to eat ad libitum. The presence of active infection with B. burgdorferi is tested by ear punch cultures followed by PCR of the culture. Mice are also tested after infection by nymphal ticks and then 2, 5, 7 and 14 days after placement of the baits in cages. The baits with compound are expected to be consumed within 1-2 days, Mice will be followed for 2 weeks to check for potential regrowth of bacteria after initial suppression.

After 2 weeks, and before sacrificing the animals to culture different organs, xenodiagnosis is performed by feeding 25 larval ticks on each. Ticks are allowed to feed to completion and collected from water moats. Ticks are tested individually for acquisition of B. burgdorferi by culture and PCR. Mice are sacrificed and the skin, heart, spleen, bladder and joints are tested by culture and PCR. For determinations of effectiveness of the HygA, the infection rates in the treated and control animals are compared using the x2 test or Fisher exact test (for small sample sizes with discrete data). The primary endpoint is culture positivity per mouse (with any culture positive being counted as a positive mouse

### Example 9: Binding of HygA to E. coli Ribosomes

One of the major concerns of a reservoir-targeted antibiotic approach is the development of resistance—or, in the case of a non-human antibiotic, cross-resistance—to a human antibiotic. Resistance remains a particular concern for a reservoir targeted antibiotic as uptake is likely to be inconsistent by infected mice resulting in periods of subtherapeutic dosing that could select for the development of resistance over time.

HygA targets the conserved peptidyl transferase center (PTC) of bacterial ribosomes.

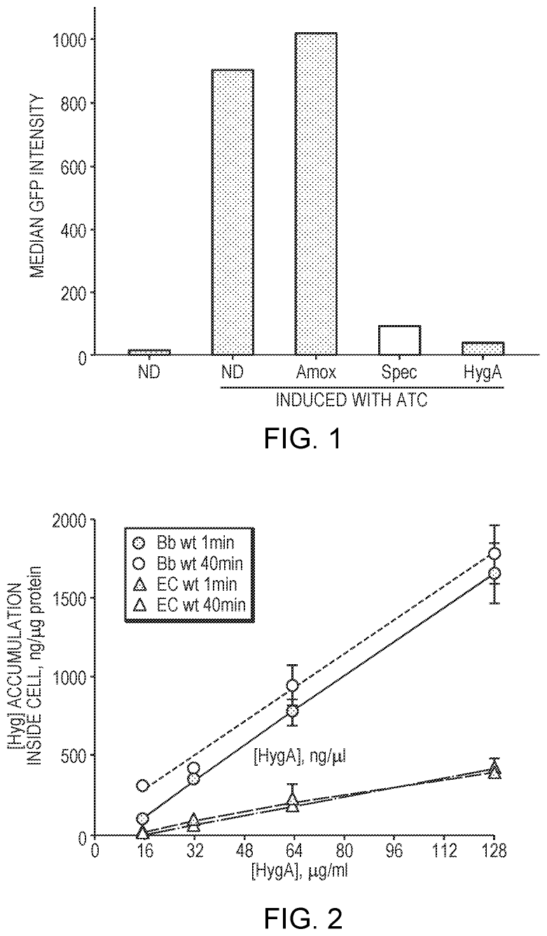

To confirm the effect of hygromycin A on protein synthesis, a GFP expressing B. burgdorferi pCRW53 was used. Cells were treated with hygromycin A or a known protein synthesis inhibitor (spectinomycin), and GFP production was then induced with anhydrotetracycline (ATC). Cells with impaired protein synthesis are expected to remain non-fluorescent. Uninduced cells, non-treated cells or cells treated with the cell wall acting amoxicillin served as a control. GFP signal was then measured for 100,000 cells by flow cytometry. Cells treated with hygromycin A had a dramatically lower level of GFP as compared to a control, showing that hygromycin A inhibits protein synthesis. (FIG. 1).

The possibility that HygA specificity could be due to a higher affinity to B. burgdorferi ribosomes was examined. HygA bound to the Thermus thermophilus ribosome showed interaction of the compound with the conserved 23S RNA nucleotides that form the core of the peptidyl transferase active site. The identity of these nucleotides was found to be the same in Borrelia, based on analysis of the genome sequence of B. burgdorferi. Next, the action of HygA on ribosomes of E. coli, which is resistant to HygA was also tested. During in vitro translation of the model ORFs, the complete arrest of translation was observed even at the lowest concentration of HygA tested (0.6 μM), which roughly approximated the concentration of active ribosomes in the reaction mixture. Under these conditions, essentially all the ribosomes were captured at the start codons of the ORFs, indicating binding of HygA to the ribosome with a very high affinity (Kd<10 nM). Since it is not possible to improve 1:1 binding, this suggests that the basis for selective action of HygA is likely not due to binding to ribosomes.

### Example 10: Uptake of HygA by B. burgdorferi and E. coli

The role of transport of HygA into spirochetes as a possible mechanism for its selectivity was explored. Like Gram negative bacteria, spirochetes have an outer membrane that serves as an additional penetration barrier. This barrier however lacks lipopolysaccharide (LPS) and is not as efficient as the outer membrane of gram-negative species such as E. coli. In E. coli, LPS on the surface of the outer membrane forms a hydrophilic network that restricts penetration of large or hydrophobic compounds. Substances that leak through the barrier are extruded by trans-envelope multidrug resistant pumps (MOR). The penetration of HygA into B. burgdorferi with that of a “hyper-porinated” E. coli delta tolC-Pore. This E. coli strain carries a modified, inducible FhuA siderophore receptor that forms a large pore in the outer membrane. As a result, the outer membrane is structurally intact, but has lost its barrier function. E. coli delta tolC-Pore also carries a deletion in the TolC, which encodes a channel serving as an exit portal for MOR substrates, further increasing the permeability of this strain.

### Example 11: Mechanisms of Resistance of B. burgdorferi to HygA

Hyg A resistant mutants are identified using a wild-type strain of B. burgdorferi through repeated passaging in sub-inhibitory concentrations of HygA. B. burgdorferi (strain N40) is exposed to low concentrations of HygA, ½ MIC, washed, recovered in HygA-free media, and then re-exposed to HygA. Bacteria are sampled at each passage to determine MICs. Bacteria that show an increase in MIC>5 fold are stored. Whole genome sequencing of strains that show increasing resistance is performed in order to identify the mutations accounting for the resistance. This process is repeated up to 20 times or until mutants are identified mutants with a 10-fold increase in MIC. Resistant is expected to be conferred by mutations in the target, 23S rRNA, or in the putative transporter. Alternatively, the resistant mutants selected in a mutS deficient background are sequenced. For identified genes related to increased resistance to HygA, allelic replacements in a wild type background are created to test whether the phenotype correlates with the mutations. All passaged mutants and constructed strains are plasmid typed to ensure that the phenotype is not due to loss of a plasmid unrelated to the identified mutation. MICs for therapeutic agents and/or antibiotics e.g. ceftriaxone, doxycycline, amoxicillin and azithromycin is determined using the strains identified as resistant. Alternatively, chemical mutagenesis is also tested.

Resistance in two pathogens carried by Ixodes ticks, Babesia microti and Borrelia miyamotoi is also tested.

Mutations are likely in the transport genes. It is possible that mutations may occur in the target, 23S rRNA. Given that the mechanism of action of HygA is very different from the human antibiotics used for treating Lyme disease, HygA resistance is not expected to lead to cross resistance to other antibiotics.

### Example 12: Testing of HygA Resistant Mutants for Infectivity in Mice and Ticks

Mutant strains that show increased resistance to HygA are tested for their ability to infect mice and ticks. It is common for resistant mutants to lose their ability to cause disease. The ability of HygA resistant mutants to function as pathogens is tested. Groups of 5 mice, each injected with the parental, mutant or complemented mutant organisms (at least 5 strains). Cultures of ear punches or PCR are performed at 2 and 4 weeks after infection to determine the status of the infection in mice.

The ability of the HygA resistant mutant to be successfully acquired by ticks and be re-transmitted to new mice is tested. Mice infected with the parental, mutant or complemented mutant strains are used to feed larval ticks. The larval ticks are collected and allowed to molt into nymphal ticks. Twenty nymphal ticks are crushed and individually cultured in BSK media to determine the rate of successful infection of the ticks. The remainder are allowed to feed on uninfected Peromyscus mice by placing 6 ticks into a capsule on the back of the mouse. The ticks are collected after completing the feeding and tested for infection. The mice are euthanized after 3 weeks and cultures and PCRs performed on heart, bladder, ear and joint tissues.

Ticks are alternatively infected through immersion in liquid cultures of B. burgdorferi. This approach has been used successfully to infect ticks with mutant strains of B. burgdorferi that are incapable of infecting mice. Larval Ixodes ticks are placed into a culture of B. burgdorferi containing 10⁷ bacteria/ml and submerged for 30 min. The supernatant is then aspirated, and the ticks are gently washed in PBS. The ticks are allowed to feed on uninfected mice as previously described. Individual ticks are recovered after feeding and crushed for culture and PCR for B. burgdorferi. A proportion of the ticks are allowed to molt to the nymphal stage. These ticks are fed on uninfected mice and then the ticks and the mice are tested for the presence of B. burgdorferi by culture and PCR.

### Example 13: Determining Sensitivity and Potential Development of Resistance of Anaplasma phagocytophilium

In addition to B. burgdorferi, Ixodes ticks and Peromyscus mice frequently carry another major bacterial pathogen namely A. phagocytophilium. A. phagocytophilium is sensitive to some antibiotics (e.g., doxycycline) used to treat B. burgdorferi. To determine activity of HygA against A. phagocytophilium, strains NCH-1 and NTN-1 are grown in the human promyelocytic cell line HL-60 at 37° C. and 5% CO2. Cells are cultured with RPMI 1640 supplemented with 20% fetal bovine serum and 2 mM L-glutamine. The percentage of infected cells is monitored by Giemsa stained cytospin preparations. Doxycycline is used as the positive comparator and ampicillin as the negative comparator. Cell toxicity using a trypan blue staining of an aliquot and percentage of A. phagocytophilium found within cytospin preps of a duplicate aliquot are determined. Infected cells are centrifuged and the supernatant replaced to remove extracellular bacteria. Uninfected HL-60 cells are counted and then mixed with infected cells so that 5% of the cells in a culture contain A. phagocytophilium. The cell mixture is aliquoted into a 96 well plate and the compounds or controls are added at various concentrations. Plates are incubated at 37° C. in 5% CO2 for 5 days and the plates examined by phase contrast microscopy for scoring each well; wells with control compounds not affecting Anaplasma growth will have most of the HL60 cells lysed. To ensure that low level growth is not missed, at day 5 p.i. the media is replaced with fresh media without the compounds and incubated an additional 5 days, with assessment of cell viability by trypan blue staining (microscopy) as well as measuring Anaplasma growth by transferring 5 μL aliquots from each well to 12 well slides, drying, and immunostaining using a rabbit polyclonal antibody and Alexa Fluor secondary antibody (host cell counterstained with GelRed). The lowest antibiotic concentration that results in <5% infected cells without causing cell death is considered the MIC. Once the MIC is established, A. phagocytophilium is cultured with gradually increasing subinhibitory levels of HygA. After a minimum of 10 passages, MIC testing is conducted to determine whether the MIC has changed.

### Example 14: Cross-Resistance of Salmonella to Antibiotics after Serial Passage in Increasing Amounts of HygA

Salmonella is a human pathogen that is frequently carried by wild mice and so poses a specific concern for reservoir targeted antibiotic strategies. Salmonella species and strains have shown high level resistance to HygA making it unlikely that distribution of HygA will select for increased resistance (either to HygA or to other bacteria). Different strains of Salmonella typhimurium from humans and mice are serially passaged sub-MIC concentrations of HygA or vehicle including a concentration which results in approximately 50% killing. A sample of the bacteria at each passage is saved. With each passage, bacteria are exposed to a range of concentrations and the concentration that results in 50% killing compared to vehicle is selected. After 10 passages, the Salmonella are plated and MIC is determined by microtiter dilution for HygA, ceftriaxone, doxycycline, azithromycin, gentamicin and ciprofloxacin which are human approved antibiotics that have activity against Salmonella. If a change in MICs for any of the drugs from the original strain that is not also present in the vehicle passaged strains is identified, whole genome sequencing is performed to identify the genetic changes between the pre and post-passage strains.

### Example 15: Field Trial of HygA in an Area Endemic for Lyme Disease

To fully understand the effects of HygA as a reservoir targeted antibiotic a field trial is conducted. Although there are other reservoirs of B. burgdorferi including birds, chipmunks, voles, Peromyscus mice are the primary reservoir. Modeling of interventions that target the main reservoir have predicted that it is possible to decrease the carriage in other reservoirs through decreasing carriage in the main reservoir without specific interventions for the other reservoirs.

An exploratory field trial is conducted on an isolated, privately-owned island with restricted access, off the west end of Nantucket Island in Nantucket Sound. Human presence is minimal and the island is accessible only by private boat. Physiographically, the island has dense secondary successional growth of bayberry, poison ivy, greenbrier, highbush blueberry and other herbaceous plants. The fauna is nearly identical to that of Nantucket Island. Small mammals comprise white footed mice (Peromyscus leucopus); meadow voles (Microtus pennsylvanicus); short-tailed shrews (Blarina brevicauda); and jumping mice (Zapus hudsonius). The island does not have squirrels (gray or red squirrel; chipmunks) which are present on other islands. Muskrats (Ondatra zibethica) are present in marshy areas. Feral cats (Felis domesticus) are also present. Deer (Odocoileus virginianus) are the only large mammals. Foxes, coyotes, raccoons, skunk, beaver, opossum, or woodchuck are not present. Thus, the mammalian fauna that may be exposed to HygA is limited. The island has typical coastal avian species, and is known for large densities of oldsquaw, scoter, and eider ducks during colder months, but these birds are not found in mouse habitat.

Endangered or threatened avian species include short eared owls, northern harrier, roseate terns, least tern and piping plovers; the latter 3 species forage at the intertidal and the two raptors mainly prey on meadow voles.

The field trial is designed to compare B. burgdorferi transmission parameters between control and treated trapping grids. Permanently situated 0.4 hectare (=1 acre) plots consisting of 49 Longworth or Sherman live traps spaced 7.6 meters apart are arrayed in a 7×7 grid, a sampling design. A total of 6 trapping grids are established on Tuckernuck with landowner permission, with 3 serving as HygA bait deployment plots and 3 as placebo controls. The primary endpoints include (1) the proportion of mice demonstrating evidence of active infection (PCR of ear biopsy); and (2) the prevalence of infection in host-seeking deer tick adults in the fall after first bait deployment as well as that in host seeking nymphal deer ticks the following transmission season. Adult deer ticks emerge in October as a result of development from feeding nymphs the same summer; nymphs develop from larvae feeding on mice during the late summer, emerging as a single cohort the following late spring. The temporal aspects of the transmission cycle make before and after comparisons less relevant than comparisons between treatment and control plots at the same time. It is possible that delayed cumulative effects on transmission may be seen: an incremental reduction in the prevalence of infected nymphal ticks in year 2 may imply that there are fewer infected mice during that year to infect larvae, and thus promote a reduction in prevalence of infected nymphs during year 3. It is expected that HygA will be very effective at killing B. burgdorferi, and will produce measurable reductions in the force of transmission that are readily apparent and do not depend on a delayed cumulative effect.

Grids are selected so that there will be no differences in mouse density, tick density, or physiographic features. Each treatment plot is matched with a control plot approximately 500 meters away. Each month from late April through September, with sampling 30 days apart, live traps are placed and checked for 2 consecutive nights. Mice are uniquely tagged with fingerling fish tags, weighed to the nearest gram, and trap station and reproductive data is recorded. Mice are bled from the retro-orbital sinus (50-90 microliters). All ticks infesting mice are identified and counted, providing an index of infestation. A 2 mm punch biopsy will be removed from the pinna at each capture and held in 30% glycerol for PCR or culture to determine active spirochetal infection. Rhodamine B is incorporated into HygA containing bait as a marker for exposure to the baits confirming uptake of the bait independent of HygA eradicated B. burgdorferi. Ingestion of this dye appears at the follicle within 24 h and will produce a “band” of fluorescence that is readily observed as the hair continues to grow. Vibrissae (2-4) is removed and retained for evidence of rhodamine B staining. The mouse is promptly released at its station of capture. In this manner, mouse and tick density each month, age (using weight as an index), reproductive status, and prevalence are recorded.

### Example 16: Mouse Nest B. burgdorferi Infection and Status

Antibiotic baits are selectively delivered by the use of custom-made wooden nest boxes, which reduce exposure of non-target species. The nest box is entered only by climbing a 0.5 meter support stake from a space between the box and the stake, with only a 1″ hole for entry or exit. White footed mice are semi arboreal, whereas the other small mammal resident on the island (mainly voles and shrews) rarely climb. The wood construction allows for multiyear use. Mouse houses greatly reduce the possibility of accidental human contact with the baits as well as environmental contamination; the mouse houses have a hinged lid that is held in place by an eye and hook or padlock.

5 nest boxes are deployed per trapping grid, a density which is expected to provide sufficient coverage of the mouse population using the rhodamine tracking system. Mice communally nest within such boxes for extended periods and thus would be constantly exposed to bait. Once safety of HygA in the wild is established, HygA containing baits are air-dropped into a region similar to the deployment of Raboral baits for vaccination of wild-life against rabies. HygA containing baits and placebos are delivered to each nest box on a biweekly basis from the first of May until the end of September of each year. Mice generally begin reproductive activity during late May-early June and thus this delivery schedule will ensure that recruits will all be potentially treated. In addition, subadult ticks (larvae and nymphs) are only active during these times; ensuring that larvae feed on treated mice means that the resulting nymphs would not be infected to infect mice the subsequent season.

### Example 17: Efficacy of the HygA in Reducing Carriage of B. burgdorferi in Peromyscus Mice

It is expected that HygA will reduce the force of transmission in two ways: (1) eradicate infection in B. burgdorferi infected mice and (2) clear/prevent infections in ticks that feed on mice that have circulating levels of HygA. Ear punch biopsies are a relatively noninvasive means of determining active infection status because B. burgdorferi is dermotropic and easily cultivated from infected mice from very small pieces of skin. Each month, ear punches are collected from sampled mice and transported to the laboratory for DNA extraction and PCR amplification to detect B. burgdorferi using the OspA primer set. OspA is genetically stable in North American strains and the primers do not cross react with other bacteria carried by ticks. A mammalian beta actin gene target is amplified from each specimen as well to verify extraction integrity. The proportion of mice with evidence of active infection (positive PCR) is expected to decrease as mice are cumulatively exposed to HygA, whereas active infections is expected to increase among mice from the control grids. By the end of the transmission season, contingency table analysis is used to demonstrate a reduction in the prevalence of actively infected mice in the treatment grids relative to the control grids.

### Example 18: Efficacy of HygA in Reducing Carriage of B. burgdorferi in Host Seeking Ixodes Ticks

This example tests whether host-seeking ticks within and near the treatment grids are less likely to be infected than those in or near control grids, after HygA is deployed. Ticks are dragged using a standard 1 sq meter flannel cloth during biweekly bait replacements However, this dragging is limited to three 50 m random transects on each grid so as to not significantly alter the abundance of ticks. For 3 treatment grids each biweekly visit during May-July is expected to yield a minimum of 45 nymphs (total minimum sample size 270 nymphs for the 3 months); and 9 adult ticks (minimum sample size 72 for the fall and spring months). With these minimum estimates, a change from 20% infection rate to 10% in nymphs and 50% to 25% in adults in prevalence of infection between treatment and control with 80% power and alpha=0.05 with estimated baseline prevalence of 20% and 50% for nymphs and adults, respectively is expected. Sample sizes is supplemented by dragging away from near the actual experimental grids; mice resident on the outside edges of the grids may contribute ticks that seek hosts within 10-20 m of the perimeter. In addition, sampling is conducted in transects between treatment grids and their matching control grids to determine whether a gradient of infected ticks may be demonstrated as the distance increases from Hyg A is deployed. All ticks are retained in grid specific vials and returned to the lab for analysis by PCR for evidence of B. burgdorferi infection using primers for ospA. A eubacterial 16S rDNA target is used to ensure DNA extraction integrity. In addition, the prevalence of B. microti is analyzed to determine whether there are any differences in the force of transmission (entomological risk index, ERi: number of ticks per unit distance or time multiplied by the prevalence of infection, which provides the number of infected ticks per unit distance or time) of this deer tick-transmitted agent between treatment and control grids.

The differences in the prevalence of B. burgdorferi infecting host seeking nymphal ticks is not expected between treatment and control grids until year 2 after HygA bait deployment is started. The effects are expected to be seen in the nymphs from year 2 of treatment when larvae are expected to feed on mice with a reduced infection rate. Changes in the infection rate of adult ticks may be seen in the Fall of year 1 as some uninfected nymphal ticks may feed on treated mice.

Interim analyses is conducted by contingency analysis (Fisher exact test) for ticks pooled from treatment and control grids. A primary endpoint of the trial is the efficacy of HygA in reducing the prevalence of B. burgdorferi infection in host-seeking nymphal deer ticks in the first and subsequent years following vaccine deployment as this is the critical number for reducing human transmission. Efficacy is defined as the percent reduction of the prevalence of infection in ticks from trapping grids receiving vaccine bait relative to those from grids receiving placebo bait, i.e., 1-[the prevalence in ticks from vaccine grids/prevalence in control grids]. The analysis of efficacy is calculated in the next transmission season, that is, for nymphs that had fed as larvae the previous late summer/fall. A chi-square test with significance of alpha=0.05 is used to analyze the prevalence of infection in ticks from vaccine and control grids. In addition, a one-tailed 95% confidence interval is constructed around vaccine efficacy in order to assess the lower 95% confidence interval for vaccine efficacy. The oral reservoir targeted treatment approach is not likely to produce lasting effects from single use deployment but rather as a result of use every year. Withdrawing the treatments before decreasing the force of transmission below the transmission threshold would allow B. burgdorferi to reestablish itself within a transmission season or two.

### Example 19: Impact of HygA Bait Deployment on Resistance in B. burgdorferi

A subset of ear punches and ticks from HygA treated and placebo grids for B. burgdorferi are cultured. Organisms are initially grown in liquid culture and then plated to obtain single strains as mice and ticks are known to harbor multiple strains. A minimum of 10 isolates from mouse or tick samples from treatment and control plots are tested every two months for sensitivity to HygA, doxycycline and ceftriaxone. It is expected that there will be no impact on HygA sensitivity from isolates from control grids. If resistance does develop, it is expected that it would increase during years 2 and 3. Bacteria that are resistant to HygA are sequenced by a combination of Nanopore (longer reads) and Illumina miSeq (shorter reads) to allow genome assembly.

### Example 20: Experimental Methods

Described herein are the experimental methods related to Example 21, Example 22, Example 23, and Example 24.

### Strains and Growth Conditions.

Actinomycete strains were isolated as follows: soil was collected, mixed with calcium carbonate and enriched for spores by dry heat at 70° C. for 30 minutes. Cells were then serially diluted ten-fold and plated on oatmeal agar medium. After 7 days of incubation, plates were examined under a dissecting microscope and colonies with characteristic Actinomycetes morphology were re-streaked to fresh plates. After 7 days of incubation, biomass was scraped and re-suspended in TSB with 15% glycerol. Stocks were saved at −80° C. Several Actinomycetes strains were purchased from the ARS Culture Collection (NRRL). B. burgdorferi sensu stricto strain B31 (clone 5A19) was kindly provided by Monica Embers at the Tulane National Primate Research Center.

B. burgdorferi sensu stricto strain B31 5A4 NP1 GFP+(BbP1286) strain was kindly provided by Melissa Caimano at the University of Connecticut Health Center, School of Medicine. B. burgdorferi sensu stricto strain N40 (clone D10E9), B. afzelii PK serotype 2, B. garinii PBr serotype 3 and B. bavariensis Pbi serotype 4 was kindly provided by John Leong at Tufts-New England Medical Center Hospita152. B. turcica strain IST7 (DSM 16138) and Alkalispirochaeta americana strain ASpG1 (DSM 14872) was purchased from DSMZ. B. burgdorferi (strain 297; ATCC 53899), Brevinema andersonii (ATCC 43811), Leptospira interrogans serovar copenhageni (ATCC BAA-1198) and Leptospira biflexa Patoc 1 (ATCC 23582) were purchased from ATCC.

B. burgdorferi (strains B31, BbP1286, N40 and 297), B. afzelii, B. garinii, B. bavariensis, B. miyamotoi, B. turcica and B. andersonii cultures were grown in Barbour-Stoenner-Kelly II (BSKII) medium in a microaerophilic chamber (34° C., 3% 02, 5% CO2) as described previously for B. burgdorferi.

In the case of B. burgdorferi BbP1286 expressing GFP under the control of flab promoter. 50 μg/mL gentamicin and 100 μg/mL kanamycin were added as described previously.

Alkalispirochaeta americana (DSM 14872) was grown in DSMZ media 1165 as described previously at pH 9.4 in an anaerobic chamber (37° C., 5% H2, 10% CO2, and 85% N2). L. biflexa and L. interrogans were grown in ATCC medium 1470 without agar (modified Leptospira medium) containing per liter, 0.3 g Peptone (BD 211677), 0.2 g beef extract, 0.5 g sodium chloride, 10% v/v rabbit serum and 0.0012 g hemin adjusted to pH 7.4. Cultures were grown aerobically and static at 30° C.

Treponema pallidum subspecies pallidum Nichols, initially isolated from the cerebrospinal fluid of a neurosyphilis patient in Baltimore, Maryland, U.S.A. in 1912, was a gift to SJN from J. N. Miller at the UCLA Geffen School of Medicine. In vitro cultivation of T pallidum has been described in detail by Edmondson et al. Briefly, the T pallidum are grown in co-culture with rabbit epithelial cells. (Sf1Ep (NBL-11) cells (ATCC CCL-68TM)). Stocks of Sf1EP cells were between passage 36 and 40 and were maintained in Sf1Ep medium consisting of Eagle's MEM with non-essential amino acids, L-glutamine, sodium pyruvate, and 10% heat-inactivated FBS at 37° C. in air with 5% CO2. One day prior to initiation of antibiotic testing, Sf1Ep cells were seeded into tissue culture-treated 6-well cluster plates at 0.5×10⁵ per well. T pallidum cultivation medium (TpCM-2) was prepared the day before experiment initiation and pre-equilibrated in a BBL™ GasPak™ jar in which a vacuum was drawn five times (house vacuum, −12-18 um Hg). The jar was refilled with 5% CO2:95% N2 four times and a final time with 1.5% 02:5% CO2:93.5% N2. The medium was then incubated overnight in a tri-gas incubator (ThermoFisher Forma Model 3130) maintained at 34° C. and 1.5% 02:5% CO2:93.5% N2 (hereafter referred to as the low oxygen incubator). All subsequent steps in the incubation of T pallidum cultures were carried out under these conditions.

E. coli WO153 was provided by Novobiotic Pharmaceuticals.

Shigella sonnei (ATCC 25931), Salmonella enterica Typhimurium LT2 (ATCC 19585), Enterobacter cloacae (ATCC 13047), Bifidobacterium longum (ATCC BAA-999), Lactobacillus reuteri (ATCC 23272), Blautia producta (ATCC 27340) and Bacteroides fragilis (ATCC 25285) strains were purchased from ATCC. Bacteria from laboratory KLE collection were isolated as described previously. S. aureus HG003, E. coli W0153, E. coli MG1655 and P. aeruginosa P01 were grown aerobically in cation-adjusted Mueller-Hinton broth (MHIIB), all other bacteria and members of the gut flora were grown in an anaerobic chamber (5% H2, 10% CO2, and 85% N2) in Brain Heart Infusion (BHI) broth, supplemented with 0.5% Yeast Extract, 50 mM MOPS buffer, 0.1% cysteine hydrochloride and 15 μg/ml hemin (BHI-YMCH).

### Minimum Inhibitory Concentration (MIC) Assay

The microbroth dilution Minimum Inhibitory Concentration (MIC) method was used to quantitatively measure the in vitro antibacterial activity of hygromycin A against bacterial strains.

Anti-Borreliella MIC assays were performed as described previously. Briefly, cultures were grown to stationary phase and diluted 1:10 (1:100 for L. biflexa) in a 96-well plate containing Hygromycin A diluted serially 2 fold. Plates with Borrelia cultures were incubated in a microaerophilic chamber (Coy hypoxic 02 control glove box) for 7 days (34° C., 3% 02, 5% CO2) and scored visually for medium color change from pink to yellow of the phenol red present in the medium. Plates with A. americana and Leptospira cultures were grown for 4 days under anaerobic and 6 days under aerobic conditions respectively and scored visually for change in color imparted by resazurin (0.001 g/L) present in the medium. The lowest concentration of antibiotic that prevented color change was interpreted as the MIC. The samples were also observed by dark field microscopy to count the number of spirochetes and results corroborated the visual MIC score.

Three hours prior to the start of an T pallidum MIC experiment, the medium in the 6-well plates containing Sf1EP cells was removed, and the plates were rinsed with the pre-equilibrated TpCM-2 to remove traces of Sf1Ep medium and the medium replaced with 4 ml of the TpCM-2. The antibiotic to be tested was then added to each well to obtain the indicated antibiotic concentrations. Plates were then pre-equilibrated in the GasPak jar as described above and then transferred to the low oxygen incubator. Antibiotic sensitivity testing was initiated by inoculation of cultures with 3.3-3.5×10⁶ T pallidum trypsinized from an actively growing culture. Sf1Ep cultures containing antibiotics were briefly removed from the incubator, inoculated with T pallidum, pre-equilibrated, and then returned to the low oxygen incubator. Three biological replicates were used for each antibiotic concentration. Following seven days of culture the T pallidum were trypsinized to remove them from the Sf1Ep cells (Edmondson, et. al.) and quantitated by darkfield microscopy using Helber counting chambers with Thoma rulings (Hawksley, Lancing, Sussex, UK). Each culture was counted at least twice using this method. Motility of each organism was also assessed. The MIC was calculated by interpolating the yields relative to the inoculum, with the point at which the yield=inoculum represented the MIC. Hygromycin A spectrum of activity against a panel of lab strains, pathobionts and symbionts was determined as described previously. Briefly, aerobic lab strains (i.e., S. aureus HG003, E. coli W0153 and MG1655, P. aeruginosa PAO1) from liquid cultures were diluted into the assay plate to achieve 5×10 5 CFU/mL, pathobionts and symbionts from anaerobic cultures were diluted 100-fold. Assay plates were prepared by 2-fold dilution of compound across the plate and included a positive growth control. After incubating the aerobes at 37° C. for 16-20 hours and the anaerobes in an anaerobic chamber for 24-48 hours, the MIC was determined as the lowest concentration of compound that inhibits growth of the bacteria as detected by the unaided eye. All MIC assays were repeated at least in triplicate.

### The Minimal Bactericidal Concentration (MBC) Determination

The MBC of hygromycin A against B. burgdorferi was determined in 1.5 ml centrifuge tubes. Exponential phase B31 cultures (10⁷/ml) were incubated with compounds at 1×, 2×, 4× and 8=×MIC for 5 days in the microaerophilic chamber. Cells were washed 3 times with BSK-II medium, serially diluted and mixed in semi-agar plates. All the plates were incubated in the microaerophilic chamber for 20 days before counting. The lowest concentration that killed more than 3 logs of cells was determined as MBC.

### Cytotoxicity

A microplate Alamar Blue assay (MABA/resazurin) was used to determine the cytotoxicity of Hygromycin A. The cell lines used were FaDu pharynx squamous cell carcinoma (ATCC HTB-43), HepG2 liver hepatocellular carcinoma (ATCC HB-8065), and HEK293-RFP human embryonic kidney red fluorescent protein tagged (GenTarget SC007) cells, all cultured in Eagle's Minimum Essential Medium supplemented with 10% fetal bovine serum. Exponentially growing cells were seeded into a 96-well, flat bottom, tissue culture treated plate (Corning) and incubated at 37° C. with 5% CO2. After 24 hours, the medium was aspirated and replaced with fresh medium containing test compounds (2 μL of a two-fold serial dilution in water to 98 μL of media). After 72 hours of incubation at 37° C. with 5% CO2, resazurin (Acros Organics) was added to each well to a final concentration of 0.15 mM. After three hours, the A544 and A590 were measured using a BioTek Synergy H1 microplate reader to determine cell viability. Experiments were performed with biological replicates.

### Screening of Anti-Borreliella Specific Compounds

Actinomycetes were inoculated into 50 mL Falcon tubes containing 10 mL modified R5 (MR5) medium with 0.1% vitamin supplement (ATCC® MD-VS™), and incubated at 28° C. with shaking (200 rpm). After 10 days of incubation, 1 mL samples were collected and centrifuged (12,000×g, 10 min) to remove the cells. Culture supernatants (750 μl) were transferred into 96 deep well plates, and dried by centrifugal evaporator. The dried samples were resuspended in 50 μL of water, and yielded 15 times concentrated samples as compared to culture supernatant. Activity of culture extracts was evaluated against B. burgdorferi BbP1286 and counter-screened against S. aureus HG003. Activity against B. burgdorferi BbP1286 was evaluated by measuring GFP fluorescence by plate reader (Synergy™ H1, BioTek Instruments) with 485/528 nm wavelength. B. burgdorferi BbP1286 was cultured and incubated as described above. After 5 days of growth, the culture was diluted 1:100 into BSKII with 50 μg/mL gentamicin and 100 μg/mL kanamycin. In a 96-well plate, 200 μL of the diluted B. burgdorferi BbP1286 culture as added to 2 μL of extract. The plate was incubated in a microaerophilic chamber for 7 days. A reduction of 80% of the GFP signal compared to cell only control was considered as positive anti-Borrelia activity. In the case of the counter-screening, S. aureus exponential phase culture (OD600 of 0.1-0.9) was diluted to OD600 of 0.03, and evenly plated onto MHIIA plates. Concentrated samples from the actinomycetes cultures (3 μl) were spotted directly onto the bacterial lawn, and anti-Staphylococcus activity was evaluated based on the presence of a zone of inhibition.

Initial isolation of anti-Borrehella compound. S. hygroscopicus was inoculated in a 250 mL flask containing 40 mL MR5 medium with 0.1% vitamin supplement, and incubated for 7 days at 28° C. with shaking (200 rpm). A culture sample (1 mL) was separated into fractions using semi-preparative high-performance liquid chromatography (HPLC) with C18 reverse-phase column (Ultra C18 5 μm Column 250×10 mm, Restek) and eluted at a flow rate of 5 ml/min. The HPLC apparatus included a Shimadzu HPLC system equipped with an SPD-M20A diode array detector (SHIMADZU Co. Ltd., Japan). The solvent and conditions used were 0 to 5 min of 5% acetonitrile (ACN) that contained 0.1% formic acid (FA), 5 to 30 min of a linear gradient of 5 to 25% ACN that contained 0.1% FA, 30 to 31 min of a linear gradient of 25 to 100% ACN that contained 0.1% FA, and 31 to 40 min of 100% ACN. Culture sample was fractionated every 1 min, and 40 fractions were generated. Each fraction was subjected to bioassay against B. burgdorferi, and activity was observed in samples from retention time 20-21 min.

Scale up production of hygromycin A. S. hygroscopicus was inoculated in a 2 L Erlenmeyer flask containing 1 L MR5 medium with 0.1% vitamin supplement and incubated at 28° C. with shaking (200 rpm). After 10-14 days of cultivation, the culture was centrifuged, and the cell pellet was removed. The supernatant was treated with XAD16N resin (20-60 mesh, Sigma-Aldrich) to bind the active compound, and incubated overnight with agitation. After discarding supernatant, the active fraction containing hygromycin A was eluted from XAD16N resin with 1 L 100% methanol. The methanol extract was dried using a rotary evaporator, and the sample was dissolved in MilliQ water. The sample was then subjected to preparative HPLC with a C18 reverse-phase column [Luna® 5 μm C18(2) 100 Å, LC Column 250×21.2 mm, Phenomenex] and eluted at a flow rate of 10 ml/min. The solvent and conditions used were 0 to 5 min of 7% ACN that contained 0.1% FA and 5 to 43 min of a linear gradient of 7 to 15.5% ACN that contained 0.1% FA. Active compound was eluted as single peak at retention time 40 min (HPLC peak at 215 nm with purity of approximately 90%).

### Mass Spectrometry and Structure Elucidation

The HRMS and molecular formula of the active compound were determined by LC-MS/MS analysis on an LTQ Orbitrap XL™ Hybrid Ion Trap-Orbitrap Mass Spectrometer (Thermo Scientific) in positive ion mode coupled with an UltiMate 3000 RSLCnano System chromatography (Dionex). The HPLC purified active fraction was prepared in a concentration of 100 μg/mL in water with 0.1% (v/v) formic acid. The solution was separated at 0.2 μL/min on a capillary column (150 mm by ID 75 μm) packed with C18 2.5 μm resin (XSelect® CSH C18) under water:acetonitrile containing 0.1% (v/v) formic acid starting with 98:2 for 5 minutes followed by 50:50 for 20 minutes. The ion peak in positive mode m/z 512.1763 for C23H30NO12 [M+H]+(calculated 512.1768 for C23H30NO12) was determined by full-scan high resolution electrospray ionization mass spectroscopy analysis, revealing C23H29N012 as the molecular formula.

The compound of molecular mass of 511.17 Da determined by mass spectrometry was a match to hygromycin A in commercially available databases (AntiBase, Wiley). For further structural elucidation, 1H, 13C, and various two-dimensional NMR techniques, including 1H-1H correlation spectroscopy (COSY), heteronuclear multiple bond correlation (HMBC), and heteronuclear single quantum correlation (HSQC) were recorded on a 500 MHz on a Varian Inova spectrometer. Chemical shifts were in accordance with those previously reported.

### Measurement of Protein Synthesis Inhibition

Exponential cultures of the GFP-inducible B. burgdorferi strain pCRW5318 were treated with antibiotics at 2×MIC overnight (amoxicillin 0.12 ug/ml, spectinomycin 4 ug/ml, hygromycin A 0.5 ug/ml). Cultures were then induced with 5 μg/ml anhydrotetracycline overnight. No-drug cultures and uninduced cultures were used as controls. Cells were washed and analyzed using a BD FACSAria II flow cytometer with a 70-μm nozzle. B. burgdorferi cells were gated by size using forward scatter (FSC) and side scatter (SSC). GFP fluorescence (FITC-A) was acquired for 100,000 cells.

### Measurement of Hygromycin a Affinity to Ribosome

The ermBL template for toeprinting was generated by PCR. The resulting templates contained T7 promoter, ribosome binding site, the coding sequence and the binding site of the toeprinting primer. The ermBL ORF was generated by crossover 4-primer PCR using primers. The tnaC ORF was generated by PCR amplifying the tnaC gene from the pGF2500 plasmid using the primers T7-tnaC2. The toe-printing analysis of drug-dependent ribosome stalling was carried out as described60 numbering check. Briefly, the DNA templates (0.1 μmol) were transcribed and translated in a total volume of 5 μL of PURExpress (New England Biolabs, cat #E6800) reactions containing 10 pmol of E. coli ribosomes. Samples were incubated for 30 min at 37° C., followed by addition of the [32 P]-labelled NV1 toe-printing primer. The primer was extended by reverse transcriptase for 10 min and the reaction products were analyzed in sequencing gels. Gels were exposed overnight to the phosphorimager screens and scanned on Typhoon phosphorimager (GE).

The HygA-free ribosomes that escaped translation arrest at the start codon would be arrested at the downstream ‘trap’ codon because the presence of the Thr-tRNA synthetase inhibitor, borrelidin, depletes the system from Thr-tRNA.

Isolation of Hygromycin a Resistant Mutants of B. burgdorferi

Plating was used to obtain CFU counts. BSK 1.5× medium was prepared as by Samuels (Samuels, D. S. (1995). Methods Mol Biol 47, 253-259; the contents of which are herein incorporated by reference in its entirety). B. burgdorferi B31 cells (6×10⁷) were plated onto semi-solid BSKII medium containing 2×, 4×, and 8×MIC hygromycin A. After a 4-week incubation, 12 colonies were isolated from the 2×MIC plate. The colonies were picked and used to inoculate liquid BSKII medium without hygromycin A to eliminate the effect of transient resistance. This strain was once again challenged with Hygromycin A as described above. After this passage strains were regrown in liquid BSKII without hygromycin A, and the MIC of hygromycin A was checked.

### Whole Genome Sequencing

Genomic DNA from KLEx1 and KLEx2 was extracted using the QIAgen DNeasy Blood and Tissue Kit per the manufacturer's instructions. DNA samples were sent to Omega Bioservices for library prep and sequencing using the Illumina Mi-Seq platform. Geneious was used to map parental Borreliella strains to reference genomes from the National Center for Biotechnology Information, and then to map resistant mutants to parental strains. Using Geneious, polymorphisms between resistant mutants and parental strains were also mapped.

### RNAseq

The B. burgdorferi hygromycin A resistant mutant KLEx2 (resistant at 16×MIC HygA, MIC=0.25 ug/ml, 16×MIC=4 ug/ml) was grown to late exponential cultures in 500 ml BSK-II, in the presence or absence of 4 μg/ml hygromycin A. Treated and untreated cultures grew comparably and reached same cell density (as confirmed by dark-field microscopy). Cells were washed 4× in cold PBS, treated with RNA protect (QIAGEN) according to manufacturer's instructions, and resuspended in Trizol (Ambion). RNA was isolated using the RNA isolation kit from QIAGEN following the manufactures protocol. Genewiz (South Plainfileld, NJ) carried out further steps including quality control, DNAse treatment, rRNA depletion, RNA fragmentation, library preparation, and Illumina HiSeq2x150 bp sequencing. Data analysis was performed by Genewiz by trimming and mapping reads to assess differential gene expression.

Construction of B. burgdorferi Overexpressing bmpD

bmpD was amplified from B. burgdorferi B31 genome with primers NdeI bmpD (gcgCATATGTTAAAAAAAGTTTATTATTTTTTAATTTTTTTATTTATTGTTGC; SEQ ID NO: 1) and bmpD XhoI (ggcCTCGAGTTAATTTTCCATTTGCAAAACAAAGTTATCATAAGATACCTTGTC; SEQ ID NO: 2), introducing NdeI and XhoI restriction sites to the 5′ and 3′ end. The B. burgdorferi shuttle vector pJSB27527, containing a codon-optimized lad repressor transcribed from the PflaB promoter and an IPTG-inducible T5 promoter derived from pQE30—was modified to introduce NdeI and XhoI restriction sites, yielding pJSB275m. The digested PCR fragment was then ligated into pJSB275m backbone and transformed into E. coli DH5α. E. coli transformants were selected with 50 μg/ml spectinomycin. The resulting shuttle vector pJSB275m_bmpD was verified by sequencing with primers pJSB275_up2 (ACCCGGAATAAGCAGTCAAG; SEQ ID NO:3), pJSB275 down (GCTGCCTTACAAGCCTCTAC; SEQ ID NO:4) and bmpD-internal_F (CAGGGCTTTCTGGTATAGGG; SEQ ID NO:5). 30 μg of ethanol precipitated plasmid pJSB275m_bmpD was transformed into electrocompetent B. burgdorferi cells as previously described. Transformants were selected with kanamycin (100 μg/ml) and streptomycin (50 μg/ml) by semisolid plating. To quantify growth, plasmid pJSB275m_bmpD was transformed into a B. burgdorferi strain constitutively expressing GFP; Bb128665. Bb1286 was generated from B. burgdorferi strain B31 5A4 NP1 (bbe02 disrupted with PflgB-KanR66) and carries a PflaB_gfp PflgB-aacC1 cassette inserted into the endogenous cp26 plasmid. Bb1286 transformants carrying the bmpD overexpression plasmid were confirmed by PCR and sequencing using primers pJSB275 up2, pJSB275 down and bmpD-internal_F. Plasmid content of transformants was verified by multiplex PCR as previously described67. BmpD overexpression was confirmed by RT-PCR as previously described, using primers bmpD_RT_F (GGATACTTTGCGTCGAAGGC; SEQ ID NO:6) and bmpD_RT_R (TGCATACTTAGCACCAGCTTCA; SEQ ID NO:7) and normalizing to the levels of recA.

Construction of E. coli Overexpressing bmpD

bmpD was amplified from B. burgdorferi B31 genome with primers SalI bmpD (gatcGTCGACAAGCAAGGAGGATATTTTTATGTTAAAAAAAG; SEQ ID NO:8) and bmpD_SbfI (gagaCCTGCAGGTTAATTTTCCATTTGCAAAACAAAGTTATCATAAGATACC; SEQ ID NO: 9), introducing SalI and SbfI restriction sites to the 5′ and 3′ end. The digested PCR fragment was then ligated into pBAD30 backbone and transformed into E. coli DH5α. E. coli transformants were selected with 50 μg/ml ampicillin. Transformants carrying pBAD30 bmpD were verified by sequencing with primers pBAD-F (ATGCCATAGCATTTTTATCC; SEQ ID NO:10) and pBAD-R (AGTTTATGGCGGGCGTCCTG; SEQ ID NO:11). Plasmid pBAD30 bmpD was then transformed into E. coli ΔTolC by heat shock. Transformants were selected with kanamycin (50 μg/ml) and ampicillin (50 μg/ml) and verified by sequencing with primers mentioned above.

### MIC Fold-Change and Adenosine Addition Experiment

To investigate the effect of bmpD overexpression in B. burgdorferi on the efficacy of hygromycin A, Bb1286+pJSB275m_bmpD was grown to early stationary phase in BSK-II medium containing kanamycin (100 μg/ml) and streptomycin (50 μg/ml). Cultures were diluted 1:10 in fresh BSK-II and divided into two; 1 mM IPTG was added to one culture, while the other culture remained untreated. Hygromycin A MIC was determined by standard microbroth dilution method, as described previously. Inhibition of growth was measured via GFP fluorescence by a microplate reader (emission 528 nm and excitation 485 nm). Ceftriaxone was used as a control antibiotic. The MIC of the bmpD-overexpressing B. burgdorferi strain Bb1286+pJSB275m_bmpD was normalized to the MIC of the wild-type B. burgdorferi strain (Bb1286) for each antibiotic. No growth difference was detected between B1286+pJSB275m_bmpD with or without 1 mM IPTG or as compared to B1286 wild-type, as assessed by daily GFP reads over the course of eight days.

To investigate the effect of bmpD overexpression in E. coli on the efficacy of hygromycin A, E. coli ΔTolC+pBAD30 bmpD was grown to mid exponential phase in M9 minimal medium with 0.2% glycerol as sole carbon source and then shifted to either 0.2% L-arabinose containing medium for inducing conditions or to 0.2% D-Glucose containing medium for repressing conditions. E. coli ΔTolC carrying the empty plasmid pBAD30 was used as a control. Hygromycin A MIC was determined by standard microbroth dilution method, as described previously. Hygromycin A MIC was also assessed in rich LB medium and in the presence of different concentrations of adenosine (2.5-2500 μM) added to minimal M9 medium with inducing arabinose or repressing glucose conditions. Inhibition of growth was measured via optical density at 600 bm by a microplate reader. The hygromycin A MIC of the bmpD overexpressing E. coli strain ΔTolC+pBAD30 bmpD and of the empty plasmid carrying E. coli strain ΔTolC+pBAD30 was normalized to the hygromycin A MIC of wild-type E. coli ΔTolC for each medium type. No growth difference was detected between the strains in minimal medium under repressing or inducing conditions, as compared to the wild-type strain, or by addition of indicated concentrations of adenosine, as assessed by measuring optical density at 600 nm.

Intracellular accumulation of hygromycin A. B. burgdorferi and the antibiotic hypersusceptible E. coli strain ΔTolC-Pore 20 were grown in BSKII and MOPS-M9 (pH 7.2) media, respectively. At OD600=0.1-0.2 for E. coli and =0.01-0.02 for B. burgdorferi, 50 mL of cell cultures were pelleted by centrifugation at room temperature (RT). Cells were washed twice in MOPS-M9 and concentrated 20-fold. Cells were incubated with 16, 32, 64 and 128 μg/mL of antibiotics and after 1 and 40 min incubation at room temperature, 100 μL cell aliquots were collected by vacuum filtration onto 1.0 μm Glass Fiber Type B filters. Filters were washed twice with 10 mM Tris-HCl (pH 8.0), dried and placed into 100% methanol at −80° C. for at least 10 min. Intracellular material was extracted by water bath sonication for 1 min. Cell and filter debris were separated by ultracentrifugation at 100,000×g for 8 min at 16° C. and the pellet re-extracted with 80% methanol in water by sonication for 15 min followed by ultracentrifugation at the same conditions. Supernatants from two extractions were combined, evaporated to dryness under vacuum and resuspended in 60 μL of 100% methanol. For compound quantification, 5 μL of solution was analyzed in triplicates. The calibration curve for hygromycin was generated in the same experiment by mixing antibiotic at increasing concentrations with the sonicated E. coli cell extracts.

An Agilent 1290 Infinity II ultrahigh-pressure liquid chromatography (UHPLC) system and 6545 quadrupole/time-of-flight (Q/TOF) system (Agilent Technologies) were used to quantify hygromycin. A Zorbax Rapid Resolution High Definition column (RRHD, 2.1×50 mm, 1.8 μm) was used for the separation with a flow rate of 0.65 mL/min. The initial concentration of 5% MS grade Acetonitrile was maintained for 1 min, and this was followed by a linear gradient to 80% over 3 min, and then by 100% over 1.1 min which was maintained for an additional 1.2 min. HPLC solvent mixtures contained 0.1% HPLC grade formic acid (Sigma Aldrich) to improve ionization efficiency. MS parameters were as follows: gas temperature, 325° C.; capillary voltage, 4000V; fragmentor voltage, 175V; m/z range, 50-1100; detector signal acquisition rate, 4 GHz; and spectrum storage rate, 2 s-1. MassHunter qualitative and quantitative analysis B8.0 was used to quantify the hygromycin A concentration using the calibration curve.

Hygromycin A was mixed with clarified E. coli cell lysates and analyzed by UHPLC/MS. At each concentration, the compound was injected in triplicates. The data were fit into a linear dependence and were used to calculate intracellular levels of antibiotic.

For hygromycin A accumulation in E. coli ΔtolC(pBAD30) and E. coli ΔtolC(pBAD30bmpD) cells, Rapidfire RF365 (Agilent technologies) coupled to a quadrupole time of flight (Q/TOF) 6545 mass spectrometer (Agilent technologies) was used. E. coli cells were grown in MOPS-M9 medium supplemented with 0.2% glycerol overnight, induced with 0.2% arabinose for 1 hour and concentrated 10-fold. The uptake was measured at increasing external hygromycin A concentrations (16-128 μg/ml). Intracellular material was extracted in two steps, (i) water and (ii) 50% methanol, by 96-well sonication for 2 min twice. Samples were aspirated during 500 ms into a 20 uL loop, then absorbed to a solid phase extraction C18 cartridge. Samples were washed for 3 seconds with 5 mM ammonium formate with a flow rate of 1.5 mL/min, then eluted from the cartridge to the mass spectrometer with 100% methanol containing 0.1% formic acid at a flow rate of 1.00 mL/min during 6 seconds. The system is then re-equilibrated with 5 mM of ammonium formate for 1 second. MSMS transition of 511.17 to 177.09 was used to quantify hygromycin A. MS parameters were as follows: gas temperature, 325° C.; capillary voltage, 4000V; fragmentor voltage, 175V; m/z range, 50-1100; detector signal acquisition rate, 4 GHz; collision energy 22 V and spectrum storage rate, 6.s-1. MassHunter quantitative analysis B8.0 was used to quantify the hygromycin A concentration using the calibration curve.

### Mouse Infection Model

All animal experiments were conducted according to protocols that were approved by the Institute of Animal Care and Usage Committee (IACUC) at Northeastern University (Approval #16-0619). Wild type female C3H mice (Charles River Laboratories) were infected with 10⁵ B. burgdorferi N40 cells by subcutaneous injection, with no less than 3 animals per treatment group. Ear punches were collected from each animal after 2 weeks of infection and cultured into BSK-II media to confirm infection. The infection was established for 3 weeks, and then animals were dosed twice a day for 5 days with saline or ceftriaxone (156 mg/kg) by intraperitoneal injection, Hygromycin A (10, 25, 50, 70 and 250 mg/kg) by oral gavage and 75 mg/kg by intraperitoneal injection.

qRT-PCR for In Vivo B. burgdorferi Quantification

The mouse and B. burgdorferi RNA from infected tissue was extracted using RNAeasy Mini Kit (Qiagen). cDNA was synthesized using High-Capacity cDNA Reverse Transcription Kits (Applied Biosysterms). Real-time PCR was performed with primers targeting the 16S rDNA gene using SYBR green Supermix (Bio-RAD). B. burgdorferi N40 copy number was normalized to cDNA standard curve synthesized from known copy number of B. burgdorferi cells.

### Bait Experiment

To prepare the mouse bait, 2 g of Bait formulation 3 (Food source), 0.5 g freshly ground peanut butter (Whole foods), 35 μl of green food coloring mix (Whole foods) were added into a 60 mm Petri dish. Boiling water (4 mL) was added and mixed with a spatula. The mixture was then dispensed into wells of a 96 well plate with a spatula such that about ⅔rd of the well was full. Antibiotics or vehicle were added at appropriate volume and mixed with a pipette tip. Added volume was kept below 60 μL per well. The bait was allowed to air dry/solidify for a few hours and the prepared baits were stored at 4° C. until use.

After one day of fasting, the animals were given bait once a day for 5 days containing Hygromycin A (200 mg/kg) or Doxycycline (100 mg/kg). Control groups included uninfected and infected animals, both given untreated bait. To ensure that the bait would be consumed, mice were housed individually with their mouse chow withheld during treatment. The doses used in mice were matched to the pharmacokinetic profile of humans given ceftriaxone 1 g every 12 hours (Sai Life Sciences Ltd, India) and doxycycline 100 mg every 12 hours.

Animals were sacrificed 3 days after completion of antibiotic treatment and skin (whole ear), heart, and quadricep muscle were collected. Skin was used for liquid culture, and RNA extraction. All other tissues were used for RNA extraction. The growth of B. burgdorferi in 10 day liquid cultures was detected by dark field microscopy with a 100× objective.

### Efficacy of Hygromycin a in Peromyscus Mice

Efficacy studies of hygromycin A in eradicating B. burgdorferi from infected mice were performed by infecting Peromyscus leucopus mice by subcutaneous inoculation with 10⁶ B. burgdorferi. After 3 weeks, mice were administered hygromycin A at either 50 mg/kg or 100 mg/kg or vehicle twice daily for 5 days. Ear punches were taken each day for culture in BSKII media containing rifampin and phosphomycin while the mice were treated with hygromycin A. An additional ear punch culture was performed on Day 7. Mice were sacrificed on Day 11 and the heart, ankle, and ear were taken for culture. Cultures were monitored by darkfield microscopy for 4 weeks or until positive.

Studies with hygromycin containing baits were performed by again infecting P. leucopus mice by subcutaneous infection with 10⁶ B. burgdorferi. Baits containing 8 mg of HygA (a dosage of approximately 400 mg/kg/day if the entire bait was eaten) or control were placed into the cages with mice. The uneaten portion of the baits were removed daily and weighed to determine consumption of HygA. Mice were fed baits for 10 days. Ear punch cultures were obtained at days 1, 3, 5 7, and 10 during antibiotic treatment and days 13 and day 15 after cessation of antibiotic dosing. Mice were sacrificed on day 17 and the heart, ankle and ear were taken for culture. Cultures were monitored by darkfield microscopy for 4 weeks or until positive.

Tissues were harvested from infected mice and processed for DNA using DNeasy Blood and tissue extraction kit (Qiagen). DNA was tested for the presence of B. burgdorferi DNA using primers that detect the B. burgdorferi flaB gene as previously described.

### Impact of Hygromycin a on the Murine Fecal Microbiome

Wild type female C3H mice (Charles River Laboratories) were infected with 10⁵ B. burgdorferi N40 cells by subcutaneous injection. The infection was established for 3 weeks, and then animals were dosed twice a day for 5 days with ceftriaxone (156 mg/kg) (Sigma) by subcutaneous injection, Amoxicillin (100 mg/kg) (Goldbio) by oral gavage, or Hygromycin A (50 mg/kg) by oral gavage, or were untreated (n=5 per group per experiment, 3 experiments). Fecal pellets were collected 3 days before and 3 days after antibiotic treatment and were stored at −80° C. in PBS. Sequencing of a stool pellet from each mouse from before and after treatment was performed by MR DNA (www.mrdnalab.com, Shallowater, TX, USA) on an Ion Torrent PGM. Using PCR primers, the V4 variable region of the 16SrRNA gene was amplified in a single-step 30 cycle PCR with the HotStarTaq Plus Master Mix Kit (Qiagen, USA). The following conditions were used: 94° C. for 3 minutes and 30 cycles of 94° C. for 30 seconds, 53° C. for 40 seconds and 72° C. for 1 minute, then a final elongation step for 5 minutes at 72° C. These data were analyzed with a proprietary analysis pipeline (MR DNA, Shallowater, TX, USA). Barcodes and primers and then sequences<150 bp were removed. Further, sequences with homopolymer runs exceeding 6 bp and sequences with ambiguous calls were removed. The sequences were then denoised, OTUs were generated, and chimeras were removed; OTUs were clustered at 3% divergence. Taxonomic classification was performed using BLASTn against a database derived from RDPII available at www.rdp.cme.msu.edu) and NCBI (www.ncbi.nlm.nih.gov). The change in relative abundance of the most abundant genera from before to after treatment of each individual mouse was reported. Further, raw sequences were processed in Qiita (Gonzalez, A., et al. (2018). Nat Methods 15, 796-798; the contents of which are herein incorporated by reference in its entirety) and were demultiplexed, trimmed to 100 bp, and closed reference OTU picking was performed against the Greengenes database at 97% similarity (McDonald, D. et al. (2012). ISME J 6, 610-618; the contents of which are herein incorporated by reference in its entirety). The alpha diversity based on the Simpson index was calculated in Qiita. The change in the Simpson index was calculated for the fecal microbiome of each mouse from before to after treatment. Statistical significance was calculated using a one-way ANOVA with Tukey's multiple comparisons test (p<0.05).

### Example 21: Hygromycin Transporter

To measure penetration of HygA, B. burgdorferi and E. coli cells were incubated with the antibiotic, samples were withdrawn, rapidly filtered, lysed, and intracellular concentration of the compound was determined by LC/MS. There was little penetration of the compound into E. coli (FIG. 2).

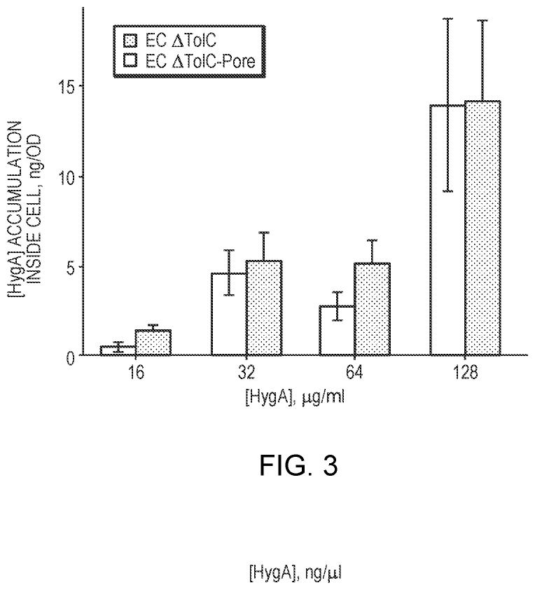

Importantly, there was no difference in penetration between cells with an intact outer membrane and those expressing the FhuA pore, suggesting that the inner membrane of E. coli is the barrier for HygA (FIG. 3 and Table 3).

TABLE 3
MIC of E. coli mutant strains

| E. coli strain | Description | HygA (ug/ml) |
|---|---|---|
| WT | BW25113 | 512 |
| ΔtolC | no efflux | 16 |
| WT-Pore | hyperporinated | 512 |
| ΔtolC-Pore | No efllux, hyperporinated | 16 |

Steady-state levels in the cytoplasm were not reached after 40 minutes of incubation even with the highest level of the compound tested, 128m/ml, suggesting that the process is very slow. By contrast, accumulation of HygA into cells of B. burgdorferi was rapid and reached saturation at 64 μg/ml (note that the relatively high levels of antibiotic in this experiment were used since a dense culture of cells is required to measure the level of intracellular compound). No significant difference in the levels of accumulated HygA could be seen after a 1 min incubation and an incubation for 40 min. Thus, HygA permeates B. burgdorferi cells rapidly, and its accumulation is fast and saturable. It is possible that HygA is transported into B. burgdorferi by a transporter that is unique to spirochetes.

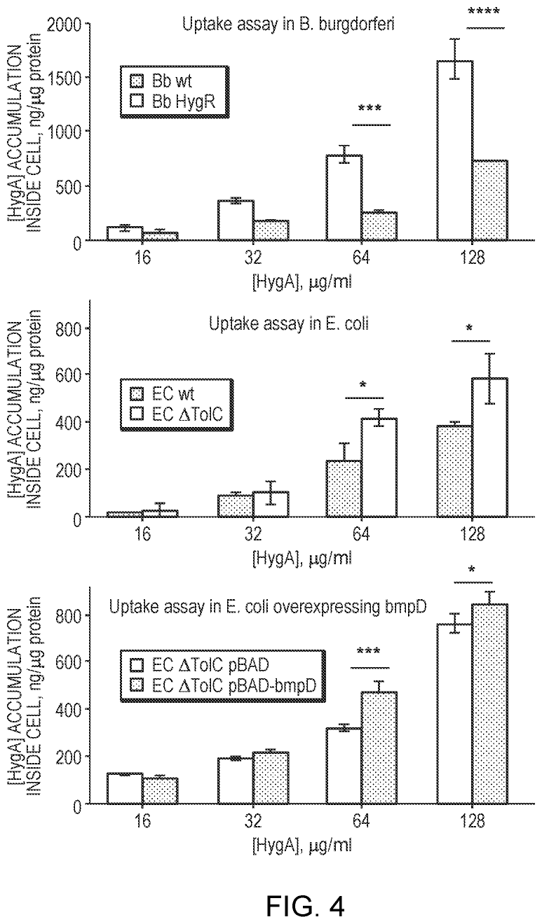

In agreement with this, efflux-deficient E. coli ΔtolC cells accumulate higher levels of hygromycin A (FIG. 4).

By contrast, accumulation of hygromycin A into cells of B. burgdorferi was rapid and substantial (note that the relatively high levels of antibiotic in this experiment were used since a dense culture of cells is required to measure the level of intracellular compound). No significant difference in the levels of accumulated hygromycin A could be seen after a short 1 min incubation and a long incubation for 40 min. Apparently, hygromycin A is smuggled into B. burgdorferi by a transporter that is unique to spirochetes and is responsible for the selectivity of the compound against this group of microorganisms.

In order to identify a possible transporter protein that transports Hygromycin A in to the cell, mutants resistant to hygromycin A were utilized. A standard approach to obtain resistant mutants was used by embedding a large number of cells (10⁸-10⁹) in semi-solid BSK medium containing various concentrations of hygromycin A. Colonies growing on hygromycin A containing plates were then picked, grown in liquid medium without antibiotic, and plated again on medium with a higher concentration of compound. An evolutionary selection method by serial passaging in increasing concentration of hygromycin in liquid cultures was also used. For both approaches, the following species were used: B. burgdorferi B31 wild-type, a B. burgdorferi hyper-mutator strain with a deletion in mutS, a fast-growing Borreliella species (B. turcica), a pooled transposon library of B. burgdorferi and Ethyl methyl sulphate mutagenized B. burgdorferi. For the evolutionary approach, cultures growing at the highest hygromycin A concentration were reinoculated in liquid medium without antibiotic and then challenged with higher concentrations of the compound. This approach however produced slow growing cells. It is known that frequent passaging of B. burgdorferi under laboratory conditions leads to plasmid loss and slow growth. In addition, many resistant isolates were unstable and lost resistance after growth in the absence of hygromycin A. Combined, this very large effort yielded only two mutants: B. turcica KLEX1 and B. burgdorferi B31 KLEX2 with stable hygromycin A resistance, MICs of 8 μg/ml and 4 μg/ml, respectively, which is a 16-fold increase as compared to their wild type parent strains. The two hygromycin A resistant mutants were then sequenced. KLEX1 and KLEX2 isolates carried the same two mutations in the gene coding for the 23S rRNA, the known target of hygromycin A (2629G>T and 2618G>T) (Table 4 and Table 5). Polymorphisms found in B. turcica KLEx1 as compared to parental strain. All deletions and insertions are single-base. In Table 4 and Table 5, “N/A” indicates a change that does not code for a protein and “*” indicates that the protein is not functional in both the parental and the mutant strain.

TABLE 4
Polymorphisms found in B. turcica KLEx1

| Locus Tag | Description | Polymorphism Type | Protein Effect |
|---|---|---|---|
| DB313_RS00990 | RelA/SpoT Family CDS | SNP | Substitution |
| DB313_RS02155 (23S) | 23S rRNA gene | SNP | N/A |
| DB313_RS03380 | Alpha/beta fold hydrolase | Deletion | Frame Shift |
| DB313_RS04445 | Hypothetical Protein | SNP | Substitution |
| DB313_RS04455 | Hypothetical Protein | Substitution | Substitution |
| DB313_RS04455 | Hypothetical Protein | SNP | Substitution |
| DB313_RS04455 | Hypothetical Protein | SNP | Substitution |
| DB313_RS04480 | DUF871 domain-containing protein | SNP | Substitution |
| DB313_RS04480 | DUF871 domain-containing protein | SNP | Substitution |

TABLE 5
Polymorphisms found in B. burgdorferi KLEx2

| Locus Tag | Description | Polymorphism Type | Protein Effect |
|---|---|---|---|
| BB_0059 | CBS domain-containing protein | SNP | Substitution |
| BB_0067 | peptidase | SNP | Substitution |
| BB_0304 | UDP-N-acetylmuramoyl-tripeptide--D- | Insertion | Frame Shift |
| | alanyl-D-alanine ligase | | |
| BB_0336 | hypothetical protein | Deletion | Frame Shift |
| BB_0356 | Conserved degenerate gene | Deletion | * |
| BB_0357 | Conserved degenerate gene | Insertion | * |
| BB_0411 | Conserved degenerate gene | Insertion | * |
| BB_0412 | Conserved degenerate gene | Insertion | * |
| BB_0413 | Conserved degenerate gene | Insertion | * |
| Intergenic | Between BB_0413 and 0414 | Deletion | N/A |
| BB_r04 | rrIB (23S rRNA) | SNP | N/A |
| BB_0511 | Conserved degenerate gene | Deletion | * |
| BB_0511 | Conserved degenerate gene | Deletion | * |
| BB_0511 | Conserved degenerate gene | Deletion | * |
| BB_0511 | Conserved degenerate gene | Deletion | * |
| Intergenic | Between BB_0542 and fusA | Deletion | N/A |
| BB_0634 | recC | Deletion | Frame Shift |
| BB_0701 | hypothetical protein | Deletion | Frame Shift |
| BB_t14 | tRNA-Thr-2 | Deletion | N/A |
| BB_t15 | tRNA-Thr-3 | Insertion | N/A |
| BB_t17 | tRNA-Leu-4 | Insertion | N/A |

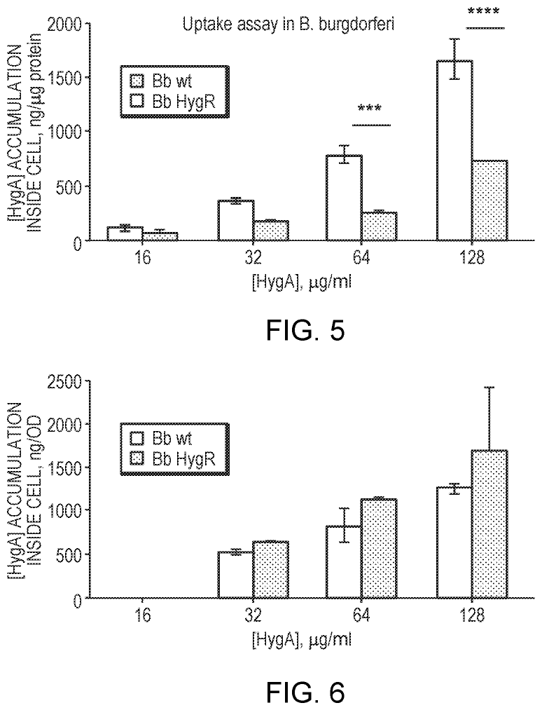

The role of additional mutations in the resistant strains which may lead to a decrease in the transport of the compound was explored. Measuring hygromycin A penetration showed that uptake of the compound was severely impaired in the B. burgdorferi KLEX2 mutant (FIG. 5), but not in the B. turcica KLEX1 mutant (FIG. 6).

The B. burgdorferi KLEX2 mutant accumulated a total of 21 polymorphisms, but no non-synonymous mutations were found in genes known to be involved in transport (Table 5). One of the genes (BB_0336) containing a frameshift mutation adjacent to an operon involved in oligopeptide transport (OppABCDF). If hygromycin A was using the oligopeptide transporter for uptake, then a mutation in BB 0336 could lead to downregulation of OppABCDF expression and antibiotic resistance. However, hygromycin susceptibility was not changed in a B. burgdorferi strain with a knockout in oppDF. This shows that the oligopeptide transport system of B. burgdorferi is not involved in hygromycin A uptake. The role of one or more of the 50 SNIPS in the KLEX2 mutant may lead to downregulation of expression of a hygromycin transporter. Such downregulation could then be detected by transcriptome analysis. Comparing the transcriptome of B. burgdorferi wild type with KLEX2, the absence of reads for many transcripts in the resistant mutant was observed which is unsurprising, because B. burgdorferi tend to lose plasmids when serially passaged in the lab. The transcriptome of KLEX2 treated with hygromycin A at 1×MIC was compared to that of untreated cells (Table 6).

TABLE 6
Differentially expressed genes in B. burgdorferi
hygromycin A resistant mutant KLEx2 in the presence
of 4 ug/ml hygromycin A, organized by P-value.

| ID | log2 Fold Change | P-value |
|---|---|---|
| 16S | −1.80 | 0.003 |
| bmpD | −1.67 | 0.005 |
| BB_H41 | −2.37 | 0.005 |
| glpK | −1.62 | 0.006 |
| BB_0183 | −1.46 | 0.010 |
| BB_J17 | 1.43 | 0.011 |
| BB_0844 | −1.41 | 0.013 |
| BB_J45 | 1.22 | 0.019 |
| BB_J31 | 1.22 | 0.020 |
| BB_0758 | 1.32 | 0.020 |
| BB_J27 | 1.18 | 0.023 |
| BB_A07 | −1.23 | 0.024 |
| BB_U05 | −1.14 | 0.025 |
| BB_0839 | −1.15 | 0.026 |
| tRNA-Cys-1 | −1.17 | 0.028 |
| BB_0212 | −1.15 | 0.030 |
| BBC10 | −8.07 | 0.032 |
| BB_0206 | 1.07 | 0.033 |
| BB_N03 | 1.15 | 0.034 |
| BB_I18 | 1.16 | 0.034 |
| BB_A34 | 1.06 | 0.034 |
| BB_P03 | 1.06 | 0.036 |
| BB_N04 | 1.06 | 0.036 |
| BB_L03 | 1.06 | 0.036 |
| BB_K42 | −1.05 | 0.039 |
| BB_J11 | 1.02 | 0.039 |
| BBC11 | −7.92 | 0.040 |
| BB_O18 | −1.04 | 0.040 |
| BB_B13 | 1.12 | 0.041 |
| BB_0731 | −1.01 | 0.048 |

Notably, the transcriptome of hygromycin A treated cells showed a 2.78-fold decrease in the expression of bmpD (UniProt ID: POCL55; SEQ ID NO: 12), a periplasmic substrate-binding protein of an ABC-type purine nucleoside transporter composed of two transmembrane proteins BB0678 (UniProt ID: 051621; SEQ ID NO: 13) and BB0679 (UniProt ID: 051622; SEQ ID NO: 14) and the ATP-binding protein BB0677 (subsequently referred to as BmpDEFG; UniProt ID: 051620; SEQ ID NO: 15). B. burgdorferi lacks enzymes required for de novo purine synthesis, and BmpDEFG is essential for nucleoside uptake. No other genes involved in transport were significantly differentially regulated.

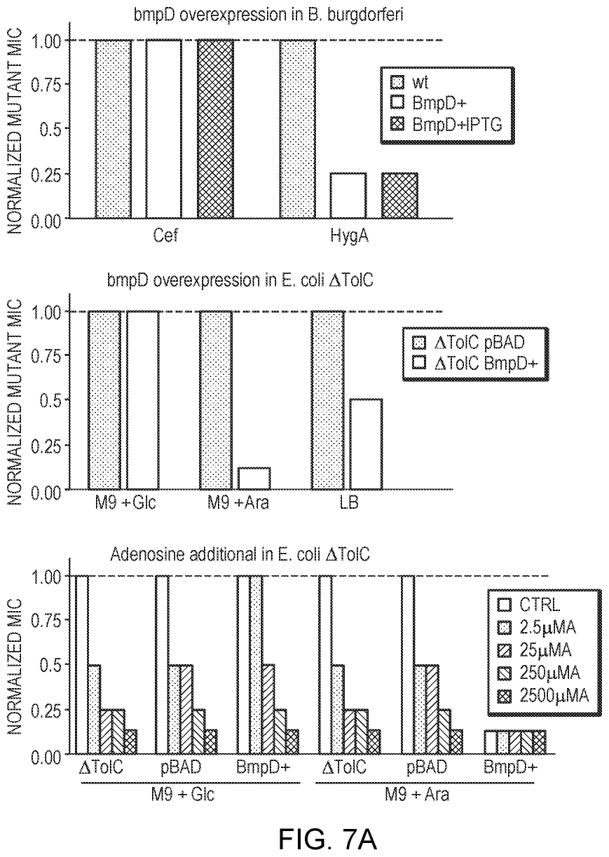

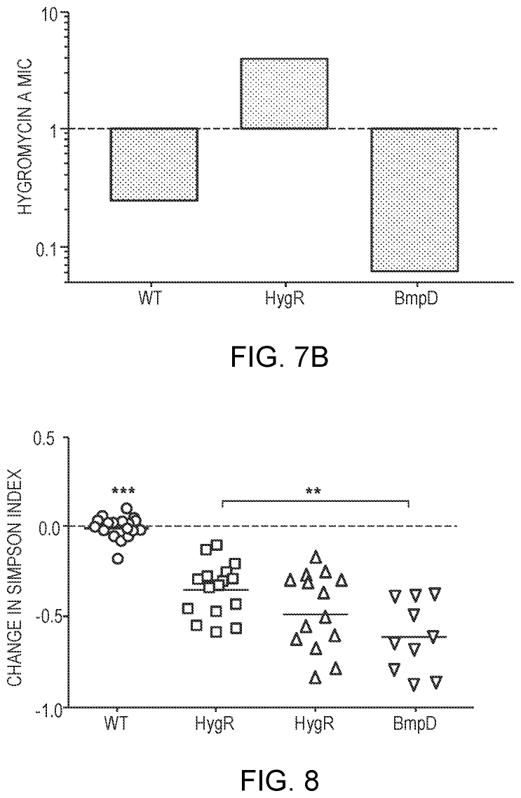

Since a decrease in expression of BmpD correlated with resistance to hygromycin A, the effect of overexpressing the protein was tested. bmpD was cloned under the control of an IPTG inducible promoter using the B. burgdorferi expression vector pJSB275m27 and assessed the MIC of this overexpression strain. Overexpression of bmpD in B. burgdorferi resulted in a four-fold reduction in hygromycin A MIC (FIG. 7A, upper panel), while the MIC for ceftriaxone remained unchanged.

This effect was IPTG-independent, likely due to leakiness of the promoter in the construct. Overexpression of BmpD had no effect on the MIC of ceftriaxone, a cell wall acting antibiotic. The ability of BmpD to affect hygromycin A resistance in a heterologous system, using E. coli as a host was tested. To this end, bmpD was cloned into the pBAD30 expression vector under an arabinose promoter and transformed into E. coli ΔtolC (carrying a deletion in the TolC component of MDR pumps, to avoid effects from efflux of hygromycin A). Inducing expression of BmpD resulted in an 8-fold reduction of E. coli hygromycin A MIC (FIG. 7A, middle panel), in good agreement with a similar result obtained with B. burgdorferi. Comparing the MIC of B. burgdorferi from wild type B. burgdorferi to Hygromycin A resistant B. burgdorferi (HygR) to BmpD overexpressing B. burgdorferi, the MIC values were calculated as 0.25, 4, and 0.0625 respectively (FIG. 7B).

Next, the penetration of hygromycin A into E. coli carrying recombinant BmpD was measured. Expression of the protein increased penetration of hygromycin A (FIG. 4, lower panel), suggesting that nucleoside transporters contribute to the uptake of hygromycin A. E. coli nucleoside transporters NupC, nupG and yetT are induced by adenosine. Addition of adenosine to E. coli resulted in a concentration-dependent decrease in MIC up to 8-fold (FIG. 7A, lower panel). When bmpD was induced, addition of adenosine did not result in a further decrease of MIC, suggesting that either the uptake of hygromycin A, the target engagement or both are saturated under these conditions.

### Example 22: Hygromycin a Therapeutic Index

Given the high potency and selectivity of hygromycin A against Borreliella, the therapeutic potential of Hygromycin A was determined. Testing hygromycin A against a number of human cell lines showed no cytotoxicity (EC50>512), giving an impressive in vitro therapeutic index of >2,000 (Table 7).

TABLE 7
Cytotoxicity and therapeutic index
of hygromycin A by alamar blue

| Cell line | IC50 (μg/ml) | Therapeutic index |
|---|---|---|
| HepG2 | >512 | >2000 |
| FaDu | >512 | >2000 |
| HEK293 | >512 | >2000 |

Hygromycin A was tested in a mouse model of acute Lyme disease. Mice were infected with B. burgdorferi N40 by subcutaneous injection, after which the pathogen propagates and spreads from the site of injection. Three weeks after inoculation, hygromycin A was delivered by intraperitoneal (IP) injection as an aqueous solution. Therapy in the mouse model aimed to emulate treatment of humans, and antibiotics were administered twice a day for 5 days. In this model, the pathogen burden is determined by culturing skin samples in a liquid medium, and performing quantitative PCR of the pathogen 16S rRNA. Culturing in liquid is a stringent test of efficacy, even 1 surviving cell of the pathogen will result in growth. After 5 days of treatment, hygromycin A cleared the infection, similarly to the ceftriaxone control (Table 8).

Oral administration of hygromycin A was tested and similar efficacy was found. There were no indications of toxicity even at very high doses.

C3H mice or Peromyscus leucopus were infected subcutaneously with B. burgdorferi N40 and after 3 weeks were treated for 5 days with hygromycin A via oral gavage, IP injection, by bait or subcutaneous, with ceftriaxone via IP injection, with amoxicillin via oral gavage, or with doxycycline via oral gavage or by bait. Peromyscus mice were treated twice a day and total daily dose (mg/kg/day) is indicated. After treatment, the presence of B. burgdorferi cells was detected by dark-field microscopy from culture of a whole ear in BSK-II media. The percentage of mice from which cultures were positive is reported.

TABLE 8
Efficacy of hygromycin A and control antibiotics against
a murine infection with B. burgdorferi.

| | Route of | | Daily Dose | |
|---|---|---|---|---|
| Mouse | Administration | Compound | (mg/kg/day) | Culture positive (%) |
| C3H | Intraperitoneal injection | Saline | 0 | 100 |
| | | Hygromycin A | 100 | 0 |
| | | Ceftriaxone | 312 | 0 |
| C3H | Oral gavage | Hygromycin A | 50 | 50 |
| | | Hygromycin A | 100 | 0 |
| | | Hygromycin A | 140 | 0 |
| | | Hygromycin A | 500 | 0 |
| | | Amoxicillin | 200 | 0 |
| | | Doxycycline | 100 | 0 |
| C3H | Bait | Hygromycin A | 100 | 0 |
| | | Doxycycline | 200 | 0 |
| Peromyscus | Subcutaneous | Hygromycin A | 100 | 100 |
| leucopus | | Hygromycin A | 200 | 0 |

### Example 23: Microbiome Analysis

Broad spectrum antibiotics can disrupt the gut microbiome. Since hygromycin A is selective against spirochetes and is inactive against many intestinal bacterial isolates (Table 9), the microbiome changes that accompany treatment of C3H mice with this compound were examined. In (Table 9), “a” indicates cultivated under microaerophilic conditions, “b” cultivated under anaerobic conditions, “c” indicates stool isolated and “KLE” indicates laboratory collection.

TABLE 9
Minimum inhibitory concentration (MIC) of hygromycin A

| | Concentration (μg/mL) | | | |
|---|---|---|---|---|
| Organism and genotype | Hygromycin A | Doxycycline | Amoxicillin | Ceftriaxone |

Spirochaetes

| | | | | |
|---|---|---|---|---|
| Treponema pallidum | 0.03 | N/A | N/A | N/A |
| Borreliella burgdorferi P1286 ^{a} | 0.12 | 0.25 | 0.12 | 0.01 |
| Borreliella afzelii ^{a} | 0.25 | 0.12 | 0.01 | <0.01 |
| Borreliella bavariensis ^{a} | 0.25 | 0.12 | 0.06 | 0.01 |
| Borreliella burgdorferi 297 ^{a} | 0.25 | 0.25 | 0.25 | 0.01 |
| Borreliella burgdorferi B31 ^{a} | 0.25 | 0.25 | 0.25 | 0.01 |
| Borreliella burgdorferi N40 ^{a} | 0.25 | 0.25 | 0.12 | 0.01 |
| Borreliella garinii ^{a} | 0.25 | 0.12 | 0.12 | 0.01 |
| Borreliella miyamotoi ^{a} | 0.25 | 0.12 | 0.12 | 0.01 |
| Borrelia turcica DSM 16138 ^{a} | 0.5 | 0.25 | 0.25 | 0.06 |
| Alkalispirochaeta americana DSM 14872 ^{b} | 0.5 | N/A | N/A | N/A |
| Brevinema andersonii ATCC 43811 ^{a} | 2 | 0.12 | 0.12 | 0.01 |
| Leptospira biflexa ATCC 23582 | 4 | 2 | 8 | 2 |
| Leptospira interrogans ATCC BAA-1198 | 4 | N/A | N/A | N/A |

Opportunistic pathogens

| | | | | |
|---|---|---|---|---|
| Fusobacterium nucleatum KLE 2428 ^{b, c} | 1 | 0.12 | 2 | 0.5 |
| Clostridium tertium KLE 2303 ^{b, c} | 8 | 0.03 | 32 | 1 |
| Clostridium perfringens KLE 2326 ^{b, c} | 8 | 0.25 | 0.12 | <0.01 |
| Staphylococcus aureus HG003 | 8 | 0.12 | 1 | 8 |
| Escherichia coli W0153 | 16 | 0.12 | 4 | 0.01 |
| Staphylococcus epidermidis KLE 2378 ^{b, c} | 32 | 0.5 | >128 | 1 |
| Shigella sonnei ATCC 25931 ^{b} | 64 | 4 | 2 | 0.25 |
| Escherichia coli MG1655 | 128 | 2 | 16 | 0.06 |
| Pseudomonas aeruginosa PAO1 | >128 | 32 | >128 | 64 |
| Salmonella typhimurium LT2 ATCC 19585 ^{b} | >128 | 8 | 16 | 0.25 |

Gut commensal bacteria

| | | | | |
|---|---|---|---|---|
| Streptococcus salivarius KLE 2370 ^{b, c} | 2 | 0.12 | 0.06 | 0.03 |
| Streptococcus parasanguinis KLE 2375 ^{b, c} | 4 | 4 | 64 | 0.06 |
| Bifidobacterium longum ATCC BAA-999 ^{b} | 8 | 0.25 | 1 | 4 |
| Bacteroides nordii KLE 2369 ^{b, c} | 8 | 0.25 | 32 | 1 |
| Bacteroides cellulosilyticus KLE 2342 ^{b, c} | 8 | 2 | 128 | 128 |
| Streptococcus sanguinis KLE 2374 ^{b, c} | 16 | 0.25 | 2 | 0.12 |
| Parabacteroides merdae KLE 2238 ^{b, c} | 16 | 0.06 | 1 | 0.25 |
| Lactobacillus reuteri ATCC 23272 ^{b} | 32 | 8 | 4 | 32 |
| Bacteroides fragilis ATCC 25285 ^{b} | 32 | 0.5 | 16 | 2 |
| Blautia producta ATCC 27340 ^{b} | 64 | 2 | 1 | 0.5 |
| Bacteroides ovatus KLE 2306 ^{b, c} | 64 | 0.01 | 0.5 | 0.02 |
| Bacteroides vulgatus KLE 2381 ^{b, c} | 64 | 1 | 128 | 128 |
| Bacteroides eggerthii KLE 2382 ^{b, c} | 64 | 0.01 | 0.5 | N/A |
| Enterococcus faecalis KLE 2341 ^{b, c} | >128 | 0.5 | 0.5 | >128 |
| Enterobacter cloacae ATCC 13047 ^{b} | >128 | 8 | >128 | 64 |
| Bacteroides xylanisolvens KLE 2305 ^{b, c} | >128 | 0.5 | 32 | 64 |

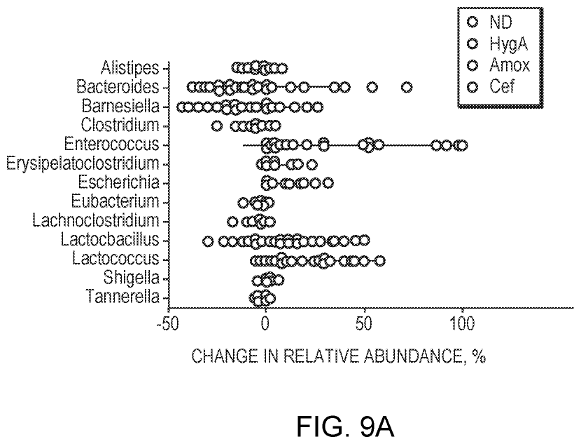

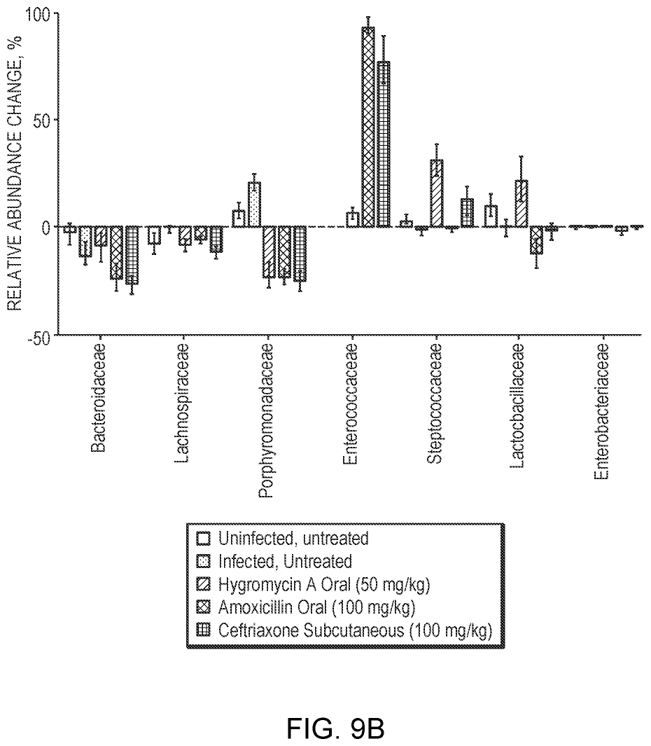

Mice were infected with B. burgdorferi N40, treated with clinically relevant doses of hygromycin A, ceftriaxone, or amoxicillin, and the microbiome composition was analyzed by sequencing 16S rDNA. Ampicillin and amoxicillin caused significant changes in the composition of the microbiota (FIG. 8, FIG. 9A and FIG. 9B).

Notably, there was a prominent bloom of pathogenic Enterococcus, resulting in 98% of total microbiome abundance in some of the animals. Amoxicillin also produced a bloom of Bacteroides in some of the mice, apparently due to the presence of resistant bacteria. By contrast, the changes produced by hygromycin were milder, with a prominent increase in symbiotic Lactobacillus and Lactococcus.

### Example 24: Hygromycin a Baits

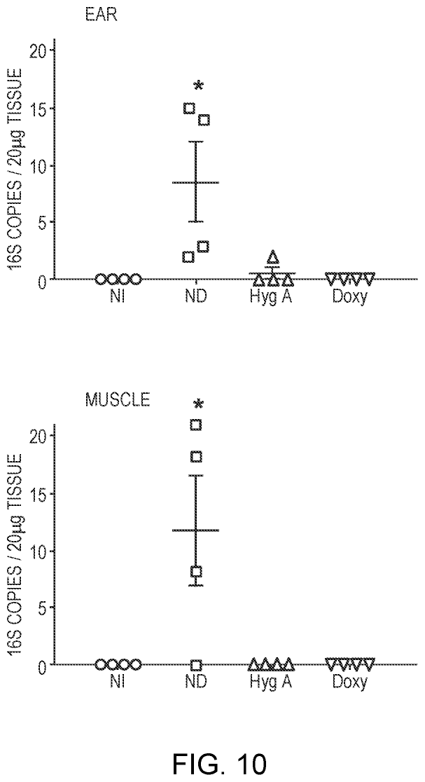

Good efficacy, safety, oral availability and selectivity of hygromycin A open an additional intriguing possibility of eradicating Lyme disease by targeting its environmental reservoir. Previously studies have shown that a bait containing doxycycline that was spread in a limited area, was able to clear the infection in 87% of mice and 94% of Ixodes ticks. This is far above levels required to reduce infection below the transmission threshold—the percentage of infected ticks and mice needed to sustain the infectious cycle in the wild. Doxycycline however is an essential part of our shrinking antibiotic armamentarium, and spreading it on large territory is unfeasible due to the risk of selecting for resistant microorganisms. Hygromycin A, with its limited activity against non-spirochetal organisms would make an ideal reservoir targeted antibiotic against B. burgdorferi. The efficacy of hygromycin A to clear B. burgdorferi by incorporating it into baits described in Example 20 was tested. Consuming baits with hygromycin A efficiently cleared B. burgdorferi infection in the animals (FIG. 10).

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art. It is further to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method of reducing Borreliella in an animal, the method comprising:
contacting a non-human animal with a Hygromycin A bait;
obtaining a tissue sample from the animal; and
measuring a level of Borreliella in the animal,
wherein the Hygromycin A bait comprises Hygromycin A at a concentration of from about 0.0001 mg/g to about 100 mg/g and wherein the contacting the animal with the Hygromycin bait reduces the level of Borreliella in the animal.

2. The method of claim 1, wherein the animal is a rodent.

3. The method of claim 2, wherein the rodent is Peromyscus spp.

4. The method of claim 1, wherein the Hygromycin A bait comprises Hygromycin A at a concentration of from about 100 mg/kg to about 1000 mg/kg body weight of the animal.

5. (canceled)

6. The method of claim 1, wherein the animal is contacted with the Hygromycin A bait for a duration from about 3 months to about 3 years.

7. (canceled)

8. The method of claim 1, wherein the Hygromycin A bait is refreshed at least biweekly.

9. The method of claim 1, wherein the animal is a tick.

10. The method of claim 9, wherein the tick is an Ixodes spp.

11. The method of claim 1, wherein the Borreliella is B. burgdorferi, B. afzelii, B. garinii, B. bavariensis or B. turcica.

12. A bait comprising Hygromycin A and a carrier, wherein the Hygromycin A is present in the bait at a concentration of about 0.0001 mg/g to about 100 mg/g.

13. The bait of claim 12, wherein the Hygromycin A is present in the bait a concentration of about 0.001 mg/g to about 5 mg/g.

14. A method of reducing transmission of Borreliella from an animal to a human, the method comprising:
contacting the animal with the bait according to claim 12,
wherein contacting the animal with the bait reduces Borreliella levels in the animal.

15. (canceled)

16. The method of claim 14, wherein the animal is a rodent.

17. The method of claim 16, wherein the rodent is Peromyscus spp.

18. The method of claim 14, wherein the bait comprises Hygromycin A at a concentration of from about 100 mg/kg to about 1000 mg/kg body weight of the animal.

19. (canceled)

20. The method of claim 14, wherein the animal is contacted with the bait for a duration from about 3 months to about 3 years.

21. (canceled)

22. The method of claim 14, wherein the bait is refreshed at least biweekly.

23. The method of claim 14, wherein the Borreliella is B. burgdorferi, B. afzelii, B. garinii, B. bavariensis or B. turcica.

24. A method of increasing sensitivity of an organism to Hygromycin A, the method comprising:
causing ectopic expression of bmpD (SEQ ID NO. 12) in the organism; and
measuring the MIC of Hygromycin A upon ectopic expression of bmpD,
wherein the ectopic expression of bmpD (SEQ ID NO: 12) decreases the MIC, thereby increasing the sensitivity of the organism to Hygromycin A.

25. The method of claim 24, wherein the sensitivity is increased by from about 1-fold to about 10 fold.

26.-27. (canceled)
